(19) United States
(12) Patent Application Publication
(10) Pub. No.: US 2025/0312385 A1
(43) Pub. Date: Oct. 9, 2025

(54) LACHNOSPIRACEAE SPP AND RUMINOCOCCUS LACTARIS STRAINS FOR THE TREATMENT AND PREVENTION OF ALZHEIMER'S DISEASE AND AGING

(71) Applicant: INSTITUT PASTEUR, PARIS (FR)

(72) Inventors: Damien REI, BOURG LA REINE (FR); Dominique CLERMONT, Paris (FR); Christiane BOUCHIER, Paris (DE); Gérald TOUAK, Paris (FR)

(73) Assignee: INSTITUT PASTEUR, PARIS (FR)

(21) Appl. No.: 18/867,478

(22) PCT Filed: May 22, 2023
(86) PCT No.: PCT/EP2023/063674

§ 371 Date: Nov. 20, 2024

(30) Foreign Application Priority Data
May 20, 2022 (EP) 22305753.0

(51) Int. Cl. A61K 35/74; A01K 67/0278; A61P 25/28

(52) U.S. Cl. CPC A61K 35/74; A01K 67/0278; A61P 25/28; A01K 2267/0356

(57) ABSTRACT

Lachnospiraceae spp and Ruminococcus lactaris new strains of bacteria for use solely or in combination, in the treatment and prevention of memory decline in an individual, in particular declines of aging or Alzheimer's disease-related origin. Compositions, in particular, an oral composition, including the Lachnospiraceae spp and Ruminococcus lactaris strains and uses thereof.

### FIELD OF THE INVENTION

The present invention concerns new strains of bacteria, a strain belonging to the Lachnospiraceae family and a strain belonging to Ruminococcus lactaris species, for use as a medicament in particular in the treatment and/or prevention of memory deficit or decline in an individual, caused by aging or Alzheimer's disease.

Memory deficit or decline is caused by numerous diseases, disorders, or conditions, including Alzheimer's disease (AD), aging, agnosia, amnesia, traumatic brain injury, dementia, postoperative cognitive dysfunction (POCD), Attention Deficit/Hyperactivity Disorder, Parkinson disease, Huntington disease or Amyotrophic Lateral Sclerosis (ALS). Aging is characterized by a progressive functional decline where the brain undergoes profound alterations, in biological, psychological, neuroanatomical, and neurophysiological functions, which are closely tied to the associated decline in cognitive functions. Indeed, normal aging is associated with a diminution in various memory abilities in several cognitive tasks involved in both episodic memory, semantic memory, and priming of both short and long term memory (Hedden et Gabrieli, 2004). The deficits may be related to impairments seen in the ability to refresh recently processed information and to the particular sensibility of the hippocampus, a brain region that plays a major role in learning and memory in mammals, to the previously described neural changes seen in aging (Dahan et al., 2020). A mix of environmental and genetic predisposition are thought to make that a subtype of the ageing population sees an exacerbation of the course of aging and progresses toward mild cognitive impairment (MCI), a transitional state between normal aging and Alzheimer's disease (Mufson et al., 2016), and an evolution toward a declared Alzheimer's disease.

The latter is a neurodegenerative disorder affecting around 40 million people worldwide and its prevalence increases with age, affecting the memory, but also locomotion and language. Alzheimer's disease greatly impairs patients' cognitive abilities. The gut microbiota (GM), recently emerged as a key player for both healthy brain functions and in the etiology of diseases. Numerous studies have shown the link between the gut microbiota (GM) and aging/Alzheimer's disease (Holmes et al., 2020), confirming the existence of an alteration of the GM composition in aged subjects (Jeffery et al., 2016) and Alzheimer's disease patients (Cattaneo et al., 2017). For example, the aging or

Alzheimer's disease phenotype is known to be transmitted through the GM in studies where rodents were colonized through fecal matter transplantation (FMT) from aged rodents (D'Amato et al., 2020; Li et al., 2020), Alzheimer's disease mouse models (Kim et al., 2021), or from aged human subjects (Rei et al., 2021). Additionally, the Alzheimer's disease phenotype is known to be alleviated through FMT from wild-type mice donors in an Alzheimer's disease mouse model (Sun et al., 2019) and probiotic treatments in Alzheimer's disease patients were shown to be beneficial (Leblhuber et al., 2018). Effective treatments against the detrimental impact of aging and Alzheimer's disease are currently inexistant. There is therefore an urgent need to find new ways to ameliorate and/or maintain cognitive abilities and the quality of life in aged subjects and Alzheimer's disease patients.

### SUMMARY OF THE INVENTION

In one embodiment, the invention provides novel substances, in particular live biotherapeutic bacterial products, and compositions and methods using the same for use as medicaments, such as in treating and/or preventing memory deficits, including aging or Alzheimer's disease-associated memory deficits in an individual.

In one embodiment, the invention provides that specific Lachnospiraceae spp and Ruminococcus lactaris strains possess the unexpected ability to restore memory abilities in aged or Alzheimer's disease-affected individuals. As illustrated in the examples below and discussed elsewhere in this invention, the invention provides that other bacteria such as Faecalicatena contorta, Faecalibacterium prausnitzii A2-165 and Roseburia intestinalis DSM14610 strains did not possess such advantageous properties. The properties of Lachnospiraceae spp and Ruminococcus lactaris strains of the invention are shown in a mouse model of Alzheimer's disease obtained through human Alzheimer's disease-patients FMT in mice and in an aged mice model of aging.

Accordingly, the invention provides compositions comprising bacteria of at least one bacterial strain selected from the Lachnospiraceae spp and Ruminococcus lactaris strains.

In one embodiment, the invention provides compositions comprising bacteria of at least one bacterial strain selected from Ruminococcus lactaris strains.

In one embodiment, the bacteria are of any one of the strains deposited to the CNCM under the accession numbers CNCM I-5830 and CNCM I-5831, respectively. In one embodiment, the bacteria are comprised in a physiologically acceptable medium. In one embodiment, the composition further comprises a prebiotic. In one embodiment, the composition further comprises a therapeutic agent. In one embodiment, the composition further comprises another compound so as the composition is formulated for use as probiotic or animal feed. In one embodiment, the composition is administered in combination with one or more other therapies or therapeutic agents.

In another embodiment, the invention provides that bacterial strains from the family Lachnospiraceae spp and the genus Ruminococcus were able to rescue the hippocampal hypo-activity of animals with memory deficit and consequently to prevent and/or reduce the memory deficit of said animals. Such results were obtained through the fluorescent-activated cell sorting (FACS) of bacterial strains grown in anaerobic conditions from young adult healthy donors for their testing as anti-aging and Alzheimer's disease treatment and prevention agents. From the 400 sorted strains, 67 were selected based on their identity and potential and their in-vitro anti-oxidant and production of anti-inflammatory short-chain fatty-acids. Interesting candidates based on those results (data not shown) were then tested in vivo for their pro-memory and anti-Alzheimer's disease and anti-aging effect in a human Alzheimer's disease-patients FMT mouse model and in aged mice, respectively. From this newly generated bacterial strain library, two bacteria characterized as a new uncharacterized strain from the genus Butyrivibrio and named Lachnospiraceae spp, and a Ruminococcus lactaris strain, genetically different than other member of the genus, showed non expected properties. Such properties of the Lachnospiraceae spp and Ruminococcus lactaris strains have been demonstrated in the examples provided by the inventors in two different models: Alzheimer's disease-patients FM transplanted mice and aged mice. As demonstrated in the examples, the Lachnospiraceae spp and Ruminococcus lactaris strains of the invention have no negative impact in an individual suffering from age or Alzheimer's disease-related memory deficits according to the invention and are thus safe for use as medicaments in animals.

Accordingly, the invention provides a method of treatment or prevention of memory deficits in a subject in need thereof, by administering a composition comprising bacteria of one or more strains selected from Lachnospiraceae spp and Ruminococcus lactaris strains, preferably bacteria of the Ruminococcus lactaris strain, to the subject.

The invention also provides a method of treating or preventing a memory deficit caused by neurodegenerative diseases of the central nervous system in a subject in need thereof, by administering a composition comprising bacteria of one or more strains selected from Lachnospiraceae spp and Ruminococcus lactaris strains, preferably bacteria of the Ruminococcus lactaris strain, to the subject.

The invention also provides a method of treating or preventing a neurodegenerative disease of the central nervous system in a subject in need thereof, by administering a composition comprising bacteria of one or more strains selected from Lachnospiraceae spp and Ruminococcus lactaris strains, preferably bacteria of the Ruminococcus lactaris strain, to the subject.

In one embodiment, the invention provides a method of treating or preventing a memory deficit caused by neurodegenerative diseases of the central nervous system, said disease being selected from Alzheimer's disease, Parkinson disease, Huntington disease or Amyotrophic Lateral Sclerosis, in a subject in need thereof, by administering a composition comprising bacteria of one or more strains selected from Lachnospiraceae spp and Ruminococcus lactaris strains, preferably bacteria of the Ruminococcus lactaris strain, to the subject.

In one embodiment, the invention provides a method of treating or preventing a neurodegenerative disease of the central nervous system, said disease being selected from Alzheimer′ disease, Parkinson disease, Huntington disease or Amyotrophic Lateral Sclerosis, in a subject in need thereof, by administering a composition comprising bacteria of one or more strains selected from Lachnospiraceae spp and Ruminococcus lactaris strains, preferably bacteria of the Ruminococcus lactaris strain, to the subject.

In one embodiment, the invention provides a method of treating or preventing a memory deficit caused by Alzheimer's disease in a subject in need thereof, by administering a composition comprising bacteria of one or more strains selected from Lachnospiraceae spp and Ruminococcus lactaris strains, preferably bacteria of the Ruminococcus lactaris strains, to the subject.

In one embodiment, the invention provides a method of treating or preventing Alzheimer's disease in a subject in need thereof, by administering a composition comprising bacteria of one or more strains selected from Lachnospiraceae spp and Ruminococcus lactaris strains, preferably bacteria of the Ruminococcus lactaris strains, to the subject.

In one embodiment, the invention also provides a method of treating or preventing a memory deficit caused by Alzheimer's disease at different stages of the disease, in a subject in need thereof, by administering a composition comprising bacteria of one or more strains selected from Lachnospiraceae spp and Ruminococcus lactaris strains, preferably bacteria of the Ruminococcus lactaris strains, to the subject.

In one embodiment, the invention also provides a method of treating or preventing a memory deficit caused by Alzheimer's disease at the preclinical stage, in a subject in need thereof, by administering a composition comprising bacteria of one or more strains selected from Lachnospiraceae spp and Ruminococcus lactaris strains, preferably bacteria of the Ruminococcus lactaris strains, to the subject.

In one embodiment, the invention also provides a method of treating or preventing a memory deficit caused by Alzheimer's disease at the mild cognitive impairment stage, in a subject in need thereof, by administering a composition comprising bacteria of one or more strains selected from Lachnospiraceae spp and Ruminococcus lactaris strains, preferably bacteria of the Ruminococcus lactaris strains, to the subject.

In one embodiment, the invention also provides a method of treating or preventing a memory deficit caused by Alzheimer's disease at the mild dementia stage, in a subject in need thereof, by administering a composition comprising bacteria of one or more strains selected from Lachnospiraceae spp and Ruminococcus lactaris strains, preferably bacteria of the Ruminococcus lactaris strains, to the subject.

In one embodiment, the invention also provides a method of treating or preventing a memory deficit caused by Alzheimer's disease at the moderate dementia stage, in a subject in need thereof, by administering a composition comprising bacteria of one or more strains selected from Lachnospiraceae spp and Ruminococcus lactaris strains, preferably bacteria of the Ruminococcus lactaris strains, to the subject.

In one embodiment, the invention also provides a method of treating or preventing a memory deficit caused by Alzheimer's disease at the severe dementia stage, in a subject in need thereof, by administering a composition comprising bacteria of one or more strains selected from Lachnospiraceae spp and Ruminococcus lactaris strains, preferably bacteria of the Ruminococcus lactaris strains, to the subject.

In one embodiment, the invention also provides a method of treating or preventing Alzheimer's disease at different stages of the disease, in a subject in need thereof, by administering a composition comprising bacteria of one or more strains selected from Lachnospiraceae spp and Ruminococcus lactaris strains, preferably bacteria of the Ruminococcus lactaris strains, to the subject.

In one embodiment, the invention also provides a method of treating or preventing Alzheimer's disease at the preclinical stage, in a subject in need thereof, by administering a composition comprising bacteria of one or more strains selected from Lachnospiraceae spp and Ruminococcus lactaris strains, preferably bacteria of the Ruminococcus lactaris strains, to the subject.

In one embodiment, the invention also provides a method of treating or preventing Alzheimer's disease at the mild cognitive impairment stage, in a subject in need thereof, by administering a composition comprising bacteria of one or more strains selected from Lachnospiraceae spp and Ruminococcus lactaris strains, preferably bacteria of the Ruminococcus lactaris strains, to the subject.

In one embodiment, the invention also provides a method of treating or preventing Alzheimer's disease at the mild dementia stage, in a subject in need thereof, by administering a composition comprising bacteria of one or more strains selected from Lachnospiraceae spp and Ruminococcus lactaris strains, preferably bacteria of the Ruminococcus lactaris strains, to the subject.

In one embodiment, the invention also provides a method of treating or preventing Alzheimer's disease at the moderate dementia stage, in a subject in need thereof, by administering a composition comprising bacteria of one or more strains selected from Lachnospiraceae spp and Ruminococcus lactaris strains, preferably bacteria of the Ruminococcus lactaris strains, to the subject.

In one embodiment, the invention also provides a method of treating or preventing Alzheimer's disease at the severe dementia stage, in a subject in need thereof, by administering a composition comprising bacteria of one or more strains selected from Lachnospiraceae spp and Ruminococcus lactaris strains, preferably bacteria of the Ruminococcus lactaris strains, to the subject.

In one embodiment, the bacteria are of any one of the strains deposited to the CNCM under the accession number CNCM 1-5830 and CNCM I-5831, respectively. In one embodiment, the bacteria are comprised in a physiologically acceptable composition. In one embodiment, the composition further comprises a prebiotic. In one embodiment, the composition further comprises a therapeutic agent. In one embodiment, the composition further comprises another compound so as the composition is formulated for use as food. In one embodiment, the composition is formulated for use as probiotic. In one embodiment, the composition is formulated for use as animal feed.

Also, the invention provides for a composition comprising bacteria of one or more strains selected from Lachnospiraceae spp and Ruminococcus lactaris strains for use as a medicament, for treatment or prevention of memory deficits or of any other diseases mentioned above. In one embodiment, the bacteria are of any one of the strains deposited to the CNCM under the accession number CNCM I-5830 and CNCM I-5831, respectively. In one embodiment, the bacteria are comprised in a physiologically acceptable composition. In one embodiment, the composition further comprises a prebiotic. In one embodiment, the composition further comprises a therapeutic agent. In one embodiment, the composition further comprises another compound so as the composition is formulated for use as food. In one embodiment, the composition is formulated for use as animal feed.

Also, the invention provides for the use of a composition comprising bacteria of one or more strains selected from Lachnospiraceae spp and Ruminococcus lactaris strains, preferably of a Ruminococcus lactaris strain for the preparation of a medicament, for treatment or prevention of memory deficits or of any other diseases mentioned above. In one embodiment, the bacteria are of any one of the strains deposited to the CNCM under the accession number CNCM 1-5830 and CNCM I-5831, respectively, preferably of the strain CNCM I-5831. In one embodiment, the bacteria are comprised in a physiologically acceptable composition. In one embodiment, the composition further comprises a prebiotic. In one embodiment, the composition further comprises a therapeutic agent. In one embodiment, the composition further comprises another compound so as the composition is formulated for use as food. In one embodiment, the composition is formulated for use as animal feed.

Also, the invention provides a method of prevention and/or treatment of memory decline due to ageing or a memory deficit caused by a neuropsychiatric and/or neurodegenerative disease, disorder, or condition, said method comprising the administration of a composition comprising Lachnospiraceae ssp and/or Ruminococcus lactaris bacteria strains, preferably from Ruminococcus lactaris bacteria, and/or a culture extract thereof, and a carrier or excipient to a patient in need.

The invention provides that the memory deficits are caused by a neuropsychiatric disease and/or a neurodegenerative disease, disorder or condition. In some embodiments, the neuropsychiatric disease and/or a neurodegenerative disease, disorder or condition is selected from Alzheimer's disease, Parkinson disease, Huntington disease or Amyotrophic Lateral Sclerosis and/or aging. In some embodiments, the neuropsychiatric disease and/or a neurodegenerative disease, disorder or condition is selected from Alzheimer's disease and/or aging. In some embodiments, the memory deficit is caused by agnosia, amnesia, traumatic brain injury, dementia, or Attention Deficit/Hyperactivity Disorder. In some embodiments, the neuropsychiatric disease and/or a neurodegenerative disease, disorder or condition is selected from Alzheimer's disease at different stages of the disease. In some embodiments, the neuropsychiatric disease and/or a neurodegenerative disease, disorder or condition is selected from Alzheimer's disease at the preclinical stage. In some embodiments, the neuropsychiatric disease and/or a neurodegenerative disease, disorder or condition is selected from Alzheimer's disease at the mild cognitive impairment stage.

In some embodiments, the neuropsychiatric disease and/or a neurodegenerative disease, disorder or condition is selected from Alzheimer's disease at mild dementia stage. In some embodiments, the neuropsychiatric disease and/or a neurodegenerative disease, disorder or condition is selected from Alzheimer's disease at moderate dementia stage. In some embodiments, the neuropsychiatric disease and/or a neurodegenerative disease, disorder or condition is selected from Alzheimer's disease at a severe dementia stage.

In one embodiment, the invention provides that memory deficits may be measured by the fear conditioning task. In another embodiment, the memory deficits may be measured by the novelty exposure assay. In some embodiments, the memory deficit is assessed with an object recognition task. In some embodiments, verbal short-term memory is tested by digit span tasks in which the individual is exposed to numbers containing various digits for various times and asked to recall the digits sometime later. In one embodiment, nonverbal short-term memory may be tested by various motor or spatial memory tasks, such as spatial information tests. In these tasks, the subject is exposed to various motor tasks or spatial orientations and asked to recall or reconstruct them later. In one embodiment, procedural memory may be tested with various tasks such as mirror drawing tests, mirror reading tasks, weight sampling tasks, speed reading of repeated nonwords, and resolving random-dot stereograms. In one embodiment, memory deficits may be assessed using various forms of classical conditioning tasks, such as eyeblink conditioning in which a tone or light onset is paired with an air puff to the eye. In one embodiment, subjects are tested on ability to classify letter strings as grammatical or non-grammatical, among other tests. In one embodiment, declarative memory may be tested with fact recall, matching tests of various sorts, tests for retention from minutes to days or years, verbal learning and recall tasks, and many other such tests.

Also accordingly, the invention provides a method of generating a mouse model exhibiting a neuropsychiatric disease and/or a neurodegenerative disease's symptoms, said method comprising the step of transferring gut microbiota obtained from fecal samples of the neuropsychiatric disease and/or a neurodegenerative disease-affected human patients to the mouse.

In one embodiment, the invention provides a method of generating a mouse model exhibiting a neuropsychiatric disease and/or a neurodegenerative disease's symptoms, said method comprising the step of transferring gut microbiota obtained from fecal samples of the neuropsychiatric disease and/or a neurodegenerative disease-affected human patients to the mouse, the said neuropsychiatric disease and/or a neurodegenerative disease being selected from Alzheimer's disease, Parkinson disease, Huntington disease or Amyotrophic Lateral Sclerosis.

In one embodiment, the invention provides a method of generating a mouse model exhibiting Alzheimer's disease symptoms, said method comprising the step of transferring gut microbiota obtained from fecal samples of Alzheimer-affected human patients to the mouse.

The invention furthermore provides a mouse model produced by the method described above.

The invention further provides the use of the above composition for the treatment and/or prevention of memory decline due to ageing.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard, no attempt is made to show structural details of the invention in more detail than is necessary for a fundamental understanding of the invention. The description taken with the drawings making apparent to those skilled in the art how the several forms of the invention.

FIG. 1A, C illustrates the 16S rRNA sequence analysis-based phylogenic classification of the strains Lachnospiraceae spp CNCM I-5830 and Ruminococcus lactaris CNCM I-5831, respectively. FIG. 1B, D shows the 10 bacterial species with the highest proximity to the 16S rRNA gene sequence of the strains Lachnospiraceae spp CNCM I-5830 and Ruminococcus lactaris CNCM I-5831, respectively.

FIG. 2A illustrates the experimental conditions for the strain treatment of Alzheimer's disease-patients FM transplanted mice (human Alzheimer's disease-like mice) and for the analysis of its effect on memory-related measures in the fear conditioning task and novelty exposure assay. FIG. 2B illustrates the effect of Faecalibacterium prausnitzii A2-165 (F. prau. A2-165, huAD), Roseburia intestinalis DSM14610 (R. intest, DSM14610, huAD), Faecalicatena contorta 18-4 (F. contorta 18-4, huAD), Lachnospiraceae spp CNCM I-5830 (CNCM I-5830, huAD) and Ruminococcus lactaris CNCM I-5831 (CNCM I-5831, huAD) strains, on memory abilities in the fear conditioning task in the human Alzheimer's disease-like mouse model (n=7, 6, 4, 5, 4, 10 and 5 mice per group). FIG. 2C illustrates a schematic of the novelty exposure assay with representative images of the increase in the number of c-fos positive neurons in hippocampal CA1 following exposure to novelty in vehicle treated young GM donor FM transplanted mice and the effect of the strains, F. prau. A2-165 (F. prau. A2-165, huAD, nov. expo.: +), F. contorta 18-4 (F. contorta 18-4, huAD, nov. expo.: +), Lachnospiraceae spp CNCM I-5830 (CNCM I-5830, huAD, nov. expo.: +) and Ruminococcus lactaris CNCM I-5831 (CNCM I-5831, huAD, nov. expo.: +) in the human Alzheimer's disease-like mouse model on the increase in the number of c-fos positive neurons in hippocampal CA1 following the exposure to novelty compared to novelty exposed vehicle-treated huAD (veh., huAD, nov. expo.: +) and non-novelty exposed vehicle-treated huY (veh., huY, nov. expo.:−) animals (n=3, 4, 3, 4, 4, 5 and 6 mice per group). Throughout the figure, bars represent the mean SD. Fear cond., fear conditioning; nov. expo., novelty exposure. One-way ANOVA, n.s., non-significant; ** p≤0.01; *** p<0.001; **** p<0.0001. Scale bar, 100 μm.

FIG. 3A illustrates the experimental conditions for the strain treatment of aged mice and for the analysis of its effect on memory-related measures in the fear conditioning task and novelty exposure assay. FIG. 3B illustrates the effect of the Lachnospiraceae spp CNCM 1-5830 (CNCM 5830, A) or Ruminococcus lactaris CNCM I-5831 (CNCM 5831, A) compared to vehicle treatment (veh., A) and to young mice (Y), on memory abilities in the fear conditioning task in the aged mice model (n=6 mice per group). FIG. 3C illustrates the effect of the Lachnospiraceae spp CNCM I-5830 (CNCM 5830, A, nov. expo.: +) or Ruminococcus lactaris CNCM I-5831 (CNCM 5831, A, nov. expo.: +) strain treatment on the number of c-fos positive neurons in hippocampal CA1 following novelty exposure in aged mice in comparison to novelty exposed vehicle treated aged (veh., A, nov. expo.: +) and non-novelty exposed young (Y, nov. expo.:−) animals (n=4, 4, 3 and 4 mice per group). Throughout the figure, bars represent the mean SD. Nov. expo., novelty exposure assay. One-way ANOVA, n.s., non-significant; * p≤0.05; ** p≤0.01; **** p<0.0001.

### DETAILED DESCRIPTION OF THE INVENTION

### Definitions

In order for the present invention to be more readily understood, certain terms are first defined below. Additional definitions for the following terms and other terms are set forth throughout the Specification.

The practice of the present invention will employ, unless otherwise indicated, conventional methods of chemistry, biochemistry, molecular biology, immunology and pharmacology, within the skill of the art. Such techniques are explained fully in the literature.

As used in this Specification and the appended claims, the singular forms “a”, “an” and “the” include plural referents unless the context clearly dictates otherwise.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.” and “i.e.” as used herein, are used merely by way of example, without limitation intended, and should not be construed as referring only to the items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 compounds” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 compounds. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising” or variations such as “comprises” or “comprising” will be understood to imply the inclusion of a stated element, integer or step, or group of elements, integers or steps, but not the exclusion of any other element, integer or step, or group of elements, integers or steps. It is understood that wherever aspects are described herein with the language “comprising” otherwise analogous aspects described in terms of “consisting of” and/or “consisting essentially of” are also provided. The term “consisting of” excludes any element, step, or ingredient not specified in the claim. The term “consisting essentially of” limits the scope of a claim to the specified materials or steps “and those that do not materially affect the basic and novel characteristic(s)” of the claimed invention.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” may mean within one or more than one standard deviation per the practice in the art. “About” or “approximately” may mean a range of up to 10% (i.e., +10%). Thus, “about” may be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg may include any amount between 4.5 mg and 5.5 mg. Furthermore, particularly with respect to biological systems or processes, the terms may mean up to an order of magnitude or up to 5-fold of a value. When particular values or compositions are provided in the instant invention, unless otherwise stated, the meaning of “about” or “approximately” should be assumed to be within an acceptable error range for that particular value or composition.

The word “substantially” does not exclude “completely” e.g., a composition which is “substantially free” from Y may be completely free from Y. Where necessary, the word “substantially” may be omitted from the definition of the invention.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Système International de Unites (SI) accepted form. Numeric ranges are inclusive of the numbers defining the range. Unless defined otherwise, all technical and scientific terms used herein have the same meaning as commonly understood by one of ordinary skill in the art to which this invention is related. For example, Juo, “The Concise Dictionary of Biomedicine and Molecular Biology”, 2nd ed., (2001), CRC Press; “The Dictionary of Cell & Molecular Biology”, 5th ed., (2013), Academic Press; and “The Oxford Dictionary Of Biochemistry And Molecular Biology”, Cammack et al. eds., 2nd ed, (2006), Oxford University Press, provide those of skill in the art with a general dictionary for many of the terms used in this invention.

“Administering” refers to the physical introduction of an agent to a subject, using any of the various methods and delivery systems known to those skilled in the art. Exemplary routes of administration for the compositions disclosed herein include oral, rectal, intravenous, intramuscular, subcutaneous, intraperitoneal, or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intradermal, intraperitoneal, transtracheal, subcutaneous, subcuticular, intraarticular, intraspinal, epidural and intrasternal injection and infusion. In some embodiments, the composition is administered via a non-parenteral route, e.g., orally. Other non-parenteral routes include a topical, epidermal or mucosal route of administration, for example, intranasally, vaginally, rectally, sublingually or topically. Administering may also be performed, for example, once, a plurality of times, and/or over one or more extended periods.

As used herein, the terms “treatment”, “treating” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment” as used herein, also covers any treatment of memory deficit or decline, or a disease, disorder, or condition causing memory deficit or decline, in a mammal, particularly in a human, and includes: (a) preventing the disease from occurring in a subject which can be predisposed to the disease or at risk of acquiring the disease but has not yet been diagnosed as having it; (b) inhibiting the disease, i.e., arresting its development; and (c) relieving the disease, i.e., causing regression of the disease. Preferred embodiments of “treatment” are further discussed below. In some embodiments, “treating” refers to administering a therapeutic agent to a patient suspected of suffering or already suffering from memory deficit or decline. It can also refer to reducing, eliminating, or at least partially arresting, as well as to exerting any beneficial effect, on one or more symptoms of the disease and/or caused by the disease and/or its complications. “Prevention” refers to administration to a patient susceptible to, or otherwise at risk of, a particular disease. Anyone in the general population is at risk for memory deficit or decline. For example, anyone is at risk for Alzheimer's disease. Some individuals have an increased, genetic risk for memory deficit or decline (e.g., Alzheimer's disease). Prevention can eliminate or reduce the risk or delay the onset of disease. Delay of onset or progression can be measured based on standard times of disease progression in similar populations or individuals.

The term “combination” refers to either a fixed combination in one dosage unit form, or a combined administration where the compound(s) of the present invention and optionally a combination partner (e.g., another drug as explained below, also referred to as “therapeutic agent” or “agent”) may be administered independently at the same time or separately within time intervals, especially where these time intervals allow that the combination partners show a cooperative, e.g., synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination to a single subject in need thereof (e.g., a patient), and are intended to include treatment regimens in which the agents are not necessarily administered by the same route of administration or at the same time.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions. Similarly, the term “increasing” indicates any change that is higher than the original value. “Increasing”, “higher” and “lower” are relative terms, requiring a comparison between pre- and post-measurements and/or between reference standards. In some embodiments, the reference values are obtained from those of a general population, which could be a general population of patients. In some embodiments, the reference values come quartile analysis of a general patient population.

The term “living bacteria”, means that the integrity of the cells is maintained and that cellular processes occur or can occur if the bacteria are cultured in the suitable medium and conditions. A living bacterium can be re-seeded in a suitable culture medium and grow under suitable conditions. Living bacteria may be preserved before administration by freezing with liquid nitrogen, gradual freezing or lyophilization and subsequent storage, preferably at temperatures ranging from +4° C. to −80° C.

The term “phylogenetically related”, means that the bacteria strains have sequences that are at least 90%, 95%, 96%, 97%, 98%, 99%, 99.5% or 99.9% identical.

The term “neurodegenerative disease” means a disease caused by the progressive loss of structure or function of neurons, in the process known as neurodegeneration. Such neuronal damage may ultimately involve cell death. Neurodegenerative diseases can include Amyotrophic Lateral Sclerosis, multiple sclerosis, Parkinson's disease, Alzheimer's disease, Huntington's disease, multiple system atrophy, and prion diseases.

Ruminococcus is a genus of bacteria in the class Clostridia. They are anaerobic, Gram-positive gut bacteria. The bacteria strains “Ruminococcus lactaris” has recently been reclassified as Mediterraneibacter lactaris but the two classifications are equivalent in the present invention.

The novel bacterial strains mentioned in the present invention were deposited according to the provisions pursuant to the Budapest treaty. The Depositing party of the bacterial strains described and/or claimed in the present patent application and the proprietor thereof express, from the outset, their consent to make available all the above strains for the whole duration of the patent.

### Bacteria of the Invention

The invention identifies for the first time the ability of specific bacteria, namely Lachnospiraceae spp or Ruminococcus lactaris strains, to be used as medicaments, for example to treat and/or prevent age or Alzheimer's disease-related memory deficits, in an individual.

The inventors have indeed unexpectedly determined that Lachnospiraceae spp and/or the Ruminococcus lactaris strains described below exhibit the ability to prevent and/or restore memory activity in the memory related brain region of the hippocampus in individuals suffering from age or Alzheimer's disease-related memory deficits. These new strains of Lachnospiraceae spp. and of Ruminococus lactaris are part of a bacterial strain library constituted from young healthy donor fecal samples.

The bacterial strains according to the invention may prevent and/or restore age or Alzheimer's disease-related memory deficits and hippocampal activity level in an individual, and in particular suffering from age or Alzheimer's disease-related memory deficits. Said memory ability and hippocampal activity may return to a normal level, since the memory abilities observed after administration of a strain according to the invention is similar to the memory observed in mice not suffering from aging or Alzheimer's disease-related memory deficits.

### Lachnospiraceae Spp Strain of the Invention

Lachnospiraceae spp is a member of the Bacillota phylum. Bacteria of this family are common in the gastrointestinal systems of many animals including humans and are implicated in the degradation of fibers, synthesis of SCFA such as butyrate, the short chain fatty acid butyrate being very important in gut physiology, systemic functions and beneficial effects for human health (Macfarlane and Macfarlane, 2011), and in the production of microbial inhibitors.

Lachnospiraceae and notably Butyrivibrio fibrisolvens are known to have anti-inflammatory and protective effects in murine models of acute and chronic colitis, i.e., in inflammatory disorders. Indeed, oral infusion of live intact Butyrivibrio fibrisolvens MDT-1 increased the rate of butyrate production measured as the concentration in feces and alleviated the formation of aberrant crypt foci (Ohkawara et al., 2005) (colon cancer pre-stages), in dextran sodium sulphate-induced experimental enterocolitis (Ohkawara et al., 2006) and delayed and reduced the 3-methylcholanthrene-induced tumor incidence in mice (Ohkawara et al., 2007). Butyrivibrio crossotus,, another member of the Lachnospiraceae family, is a butyrate-producing bacteria, and a diminution of its presence was shown to occur during the end of life of centenarians (Luan et al., 2020), suggesting its association with longevity. Also, a lower abundance of other Lachnospiraceae spp bacteria was recently shown to be caused by higher odds of amyloid and p-tau positivity in a population of Alzheimer's disease patients (Verhaar et al., 2022). This showed a negative correlation between the presence of those bacteria and those two Alzheimer's disease pathology markers.

In an aspect of the invention, it is herein provided a novel bacterium of the Lachnospiraceae family, a representative culture of which having been deposited at the Collection Nationale de Cultures de Microorganismes (CNCM), Institut Pasteur, 25 rue du Docteur Roux, 75724 PARIS Cedex 15, on Mar. 2, 2022, under the CNCM Accession Number 1-5830, for use as a medicament.

In an embodiment, the Lachnospiraceae spp bacteria strain of the invention is phylogenetically related to Butyrivibrio proteoclasticus and Butyrivibrio hungatei bacteria strains.

In another aspect, it is provided herein the culture extract of the said bacterial strain, the said culture extract being selected from the group consisting of: the strain culture supernatants, cell debris, cell walls, and protein extracts, for use as a medicament.

As disclosed in example 1 below, the 16S rRNA gene sequence of Lachnospiraceae spp CNCM I-5830 has the sequence displayed in the example section as SEQ ID NO: 1.

In an embodiment, the invention also provides a bacterial strain that has a 16S rRNA sequence that is at least 90%, 95%, 96%, 97%, 98%, 99%, 99.5% or 99.9% identical to the 16S rRNA sequence of said bacterial strain (SEQ ID NO: 1), for use as a medicament.

### Ruminococcus Lactaris Strain of the Invention

Ruminococcus lactaris is a member of the clostridia class. Bacteria that belong to clostridial cluster IV (Ruminococcaceae) are numerically abundant in the human large intestine, typically accounting for 10-40% of total bacterial 16S rRNA sequences (Lay et al., 2005). Some members of this family are known to be keystone species in the degradation of complex carbohydrates in the human colon, meaning that they are available to produce secondary products used downstream by many other bacteria, and therefore, to condition the global gut microbial composition. Also, some members in this family were showed to aid in recovery from diarrheal disease caused by Vibrio cholerae (Hsiao et al., 2014) and reduced mortality from graft-versus-host disease following allogeneic blood/marrow transplantation (Jenq et al., 2015). Also, a lower abundance of another Ruminococcus genus member, Ruminococcus torques spp., was recently shown to be caused by higher odds of amyloid positivity in a population of Alzheimer's disease patients (Verhaar et al., 2022). This showed a negative correlation between the presence of this bacteria and this Alzheimer's disease pathology marker.

In another aspect of the invention, it is herein provided a novel bacterium of the Ruminococcus lactaris species, a representative culture of which having been deposited at the Collection Nationale de Cultures de Microorganismes (CNCM), Institut Pasteur, 25 rue du Docteur Roux, 75724 PARIS Cedex 15, on Mar. 2, 2022, under the CNCM Accession Number I-5831, for use as a medicament.

In one aspect of the invention, the Ruminococcus lactaris bacteria strain is phylogenetically related to Ruminococcus lactaris ATCC 29176.

In another aspect, it is herein provided culture extract of said bacterial strain, said culture extract being selected from the group consisting of: strain culture supernatants, cell debris, cell walls, and protein extracts, for use as a medicament.

As disclosed in example 1 below, the 16S rRNA gene sequence of Ruminococcus lactaris CNCM I-5831 has the sequence displayed in the example section as SEQ ID NO: 2.

In an embodiment, the invention also provides a bacterial strain that has a 16S rRNA sequence that is at least 90%, 95%, 96%, 97%, 98%, 99%, 99.5% or 99.9% identical to the 16S rRNA sequence of SEQ ID NO: 2, for use as a medicament.

### Other Strains

Other strains that are useful in the compositions and methods of the invention, such as derivatives of one or more of the strains deposited under accession numbers CNCM I-5830 and/or CNCM I-5831, may be identified using any appropriate method or strategy.

A derivative of the strain of the invention may be a daughter strain (progeny) or a strain cultured (subcloned) from the original. A derivative of a strain of the invention may be modified, for example at the genetic level, without ablating the biological activity. In particular, a derivative strain of the invention is therapeutically active. A derivative strain will have comparable therapeutic activity to one or more of the strains deposited under accession numbers CNCM I-5830 and/or CNCM I-5831. Preferred are strains for use in preventing and/or restoring age or Alzheimer's disease-related memory deficits and hippocampal activity level in an individual, and in particular suffering from age or Alzheimer's disease-related memory deficits.

### Compositions of the Invention

In one embodiment, the invention provides compositions comprising one or more bacterial strains of Lachnospiraceae spp and Ruminococcus lactaris of the invention. In other words, the invention provides compositions comprising either bacterial strains of Lachnospiraceae spp or bacterial strains of Ruminococcus lactaris. The invention also provides compositions comprising bacterial strains of Lachnospiraceae spp and bacterial strains of Ruminococcus lactaris. In one embodiment, the compositions further comprise a pharmaceutically acceptable carrier or excipient.

In a preferred embodiment, a composition according to the invention comprises, optionally along with a pharmaceutically acceptable carrier or excipient, novel bacteria of the Lachnospiraceae spp bacterial strain deposited to the CNCM under the accession number CNCM I-5830 or bacteria of the Ruminococcus lactaris bacterial strain deposited to the CNCM under the accession number CNCM I-5831.

In a more preferred embodiment, the composition of the invention contains, optionally along with a pharmaceutically acceptable carrier or excipient, novel bacteria of the Lachnospiraceae spp bacterial strain deposited to the CNCM under the accession number CNCM I-5830 and bacteria of the Ruminococcus lactaris bacterial strain deposited to the CNCM under the accession number CNCM I-5831.

In a particular embodiment, the compositions of the invention contain culture extract of said bacterial strains, said culture extract being selected from the group consisting of: strain culture supernatants, cell debris, cell walls, and protein extracts.

In one embodiment, the compositions according to the invention are intended for the gastrointestinal tract, in particular the gut. Consequently, a composition according to the invention is selected from an oral, rectal or parenteral composition. A composition of the invention is preferably an oral or rectal composition, more preferably an oral composition. Such composition may be in the form of a suspension, tablet, pill, capsule, granulate or powder.

Advantageously, a composition according to the invention, intended for oral administration, can be provided with a coating resistant to gastric juice, so as to ensure that the bacterial strain of the invention comprised in the said composition can pass through the stomach undamaged. The release of the bacterial strain can thus take place for the first time in the colon.

In one embodiment, the compositions of the invention comprise a carrier or excipient, preferably a pharmaceutically acceptable carrier or excipient. As used herein, the term “pharmaceutically acceptable” refers to molecular entities and compositions that do not produce an adverse, allergic or other untoward reaction when administered to a mammal, especially a human, as appropriate. A pharmaceutically acceptable excipient refers to a non-toxic solid, semi-solid or liquid filler, diluent, encapsulating material or formulation auxiliary of any type. The pharmaceutically acceptable carriers or excipients that can be used in the composition according to the invention are well known to the skilled person and may vary according to the disease to be treated and the administration route. In accordance with an embodiment of the present invention, the carriers provide an improvement of the bioavailability, the stability and/or the endurance of the bacteria. In one embodiment, the carrier or excipient improves the bioavailability, the stability and the endurance of the bacteria or their secondary metabolites.

The composition of the present invention may further contain prebiotics. Prebiotics may support the growth of probiotics before they are rendered non-replicating. “Prebiotic” means non-digestible food substances that promote the growth of health beneficial microorganisms and/or probiotics in the intestines. They are not broken down in the stomach and/or upper intestine or absorbed in the GI tract of the person ingesting them, but they are fermented by the gastrointestinal microbiota and/or by probiotics. Preferably, they may be selected from the group consisting of oligosaccharides, optionally containing fructose, galactose, mannose; dietary fibers, in particular soluble fibers, soy fibers; inulin; or mixtures thereof. Preferred prebiotics are fructo-oligosaccharides, galacto-oligosaccharides, isomalto-oligosaccharides, xylo-oligosaccharides, arabino-xylo oligosaccharides, mannan-oligosaccharides, oligosaccharides of soy, glycosyl sucrose, lactosucrose, lactulose, palatinose-oligosaccharides, malto-oligosaccharides, gums and/or hydrolysates thereof, pectins and/or hydrolysates thereof.

The compositions of the invention may be administered by any method suitable for depositing in the gastrointestinal tract, preferably the small intestine and/or the colon, of the subject to be treated. In particular, the composition can be administered by enteral or parenteral route, preferably by oral, sublingual, subcutaneous, intramuscular, intravenous, transdermal, local or rectal administration route. Preferably, the composition of the invention is administered, or is adapted to be administered, by rectal or oral route.

In an embodiment, the composition is to be administered by oral route. For oral administration, the composition may be formulated into conventional oral dosage forms such as tablets, capsules, powders, granules and liquid preparations such as syrups, elixirs, and concentrated drops. Non Toxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials, are also necessary. For example, starch, gelatin, sugars such as lactose or dextrose, and natural or synthetic gums can be used as binders. Disintegrants may also be necessary in the tablets to facilitate break-up of the tablet. Disintegrants include starches, clays, celluloses, algins, gums and crosslinked polymers. Moreover, lubricants and glidants may also be included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants. Well-known thickening agents may also be added to compositions such as corn starch, agar, natural or synthetic gums, resins, methylcellulose, sodium carboxymethylcellulose, guar, xanthan and the like. Preservatives may also be included in the composition, including methylparaben, propylparaben, benzyl alcohol and ethylene diamine tetraacetate salts.

Compositions prepared for oral administration may be in a gastro-resistant oral form allowing the active compounds contained in the composition, to pass the stomach and be released into the intestine. The material that can be used in enteric coatings includes, for example, alginic acid, cellulose acetate phthalate, plastics, waxes, shellac and fatty acids (e.g. stearic acid or palmitic acid). Compositions according to the invention may be formulated to release the active ingredients substantially immediately upon administration or at any predetermined time or time period after administration. In one embodiment, the release of the bacterial strain may take place for the first time in the upper intestinal tract. In other embodiments, the composition may be a food composition or a food supplement. By “food composition” is meant any composition comprising food ingredients such as macronutrients, micronutrients, vitamins and/or minerals. The food composition may be intended for human or animal consumption and may be a liquid, paste or solid. Some examples of food compositions include, but are not limited to dairy products such as cheese, butter, cream, yoghurt, fermented milk, ice cream, cooked products such as bread, biscuits and cakes, fruit products such as fruit juice, fruit compote or fruit paste, soy food products, starch-based food products, edible oil compositions, spreads, breakfast cereals, infant formula, food bars (e.g. cereal bars, breakfast bars, energy bars, nutrition bars), chewing gum, beverages, drinking supplements (powders to be added to a beverage). As used herein, the term “food supplement” refers to any composition which is formulated and administered separately from other foods to complement the nutritional intake of a subject, i.e., a human or an animal. This supplement may be in any suitable form well known to those skilled in the art, preferably in the form of dietetic food or oral supplementation. Food compositions of the present invention may comprise a component that is typically added to a food during manufacture, for example, a protein, carbohydrate, fat, nutrient, seasoning agents and flavoring agents. Examples of the above carbohydrate are monosaccharide, e.g., glucose, fructose and the like; disaccharides, such as maltose and sucrose, oligosaccharides and the like; and a poly saccharide, such as dextrin, sugar alcohol such as a conventional sugar and xylitol, sorbitol, erythritol, such as cyclodextrins. Examples of flavorings may be natural flavors (thaumatin, stevia extract (e.g., rebaudioside A, glycyrrhizin, etc.), and/or synthetic flavors (saccharin, aspartame, etc.). For example, when the food composition of the present invention is a drink or beverages, it may further include a citric acid, liquid fructose, sugar, glucose, acetic acid, malic acid, fruit juice and other plant extracts and the like.

In another preferred embodiment, the composition is to be administered by rectal route. Suitable rectal-route forms include, but are not limited to, suppository and enema. In particular, the bacteria may be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

In one embodiment, the compositions of the invention comprise one or more additional therapeutic agents. In one embodiment, said therapeutic agent(s) are used to treat or prevent the disease, disorder, or condition underlying the memory deficit but do not necessarily have an effect themselves on memory. In one embodiment, the disease, disorder, or condition underlying the memory deficit is selected from Alzheimer's disease, aging, agnosia, amnesia, traumatic brain injury, dementia, postoperative cognitive dysfunction, or Attention Deficit/Hyperactivity Disorder, Parkinson disease, Huntington disease, or Amyotrophic Lateral Sclerosis. In a preferred embodiment, the disease, disorder, or condition underlying the memory deficit is selected from Alzheimer's disease, agnosia, amnesia, traumatic brain injury, dementia, postoperative cognitive dysfunction, or Attention Deficit/Hyperactivity Disorder. In one embodiment, the composition further comprises a therapeutic agent used in symptomatic treatment of Alzheimer's disease, such as acetylcholinesterase inhibitors and N-methyl-D-aspartate (NMDA) receptor antagonists. In one embodiment, the composition further comprises a therapeutic agent used as etiology-based treatment of Alzheimer's disease, such as secretase inhibitors, microglia-directed treatments, amyloid binders, amyloid immunotherapy, and tau therapies, including immunotherapies. In one embodiment, the composition further comprises a therapeutic agent used as microbiota-based treatment of Alzheimer's disease, such as probiotics, prebiotics, oligosaccharides and polysaccharides, or fecal microbiota transplant.

In some embodiments, the composition comprises a microbial consortium. For example, in some embodiments, the composition comprises the Lachnospiraceae spp bacterial strain of the invention and/or the Ruminococcus lactaris bacterial strain of the invention, as part of a microbial consortium. For example, in some embodiments, the Lachnospiraceae spp bacterial strain and/or the Ruminococcus lactaris bacterial strain of the invention is present in combination with one or more (e.g., at least 2, 3, 4, 5, 10, 15 or 20) other bacterial strains from other genera with which it can live symbiotically in vivo in the intestine. For example, in some embodiments, the composition comprises the bacterial strain of Lachnospiraceae spp and/or the Ruminococcus lactaris bacterial strain of the invention in combination with a bacterial strain from a different genus. In some embodiments, the microbial consortium comprises two or more bacterial strains obtained from a GM/faeces sample of a single organism, e.g., a human. In some embodiments, the microbial consortium is not found together in nature. For example, in some embodiments, the microbial consortium comprises bacterial strains obtained from GM/fecal samples of at least two different organisms. In some embodiments, the two different organisms are from the same species, e.g., two different humans. In some embodiments, the two different organisms are an infant human and an adult human. In some embodiments, the two different organisms are a human and a non-human mammal. In alternative embodiments, compositions of the invention comprise 16, 15, 14, 13, 12, 11, 10, 9, 8, 7, 6, 5 or fewer distinct bacterial species. In certain embodiments, the composition comprises 4 or fewer distinct bacterial species. In certain embodiments, the composition comprises 3 or fewer distinct bacterial species. In certain embodiments, the composition comprises 2 or fewer distinct bacterial species. In certain embodiments, the composition comprises a Lachnospiraceae spp bacterial strain and/or a Ruminococcus lactaris bacterial strain of the invention, and no other bacterial species. In preferred embodiments, the compositions of the invention comprise a single strain of Lachnospiraceae spp and/or of the Ruminococcus lactaris bacterial species. Such compositions may comprise only de minimis or biologically irrelevant amounts of other bacterial strains or species. In some embodiments, the composition comprises “substantially” no bacteria of other species, or is “substantially free” from other species of bacteria. In some embodiments in which the composition of the invention comprises more than one bacterial strain, different species or genera, the individual bacterial strains of different species or genera may be for separate, simultaneous or sequential administration. For example, the composition may comprise all of the more than one bacterial strain, species or genera, or the bacterial strains, species or genera may be stored separately and be administered separately, simultaneously or sequentially. In some embodiments, the more than one bacterial strain, species or genera are stored separately but are mixed together prior to use.

The compositions for use in accordance with the invention may or may not require marketing approval. In certain embodiments, the invention provides the above pharmaceutical composition, wherein said bacterial strain is lyophilized. In certain embodiments, the invention provides the above pharmaceutical composition, wherein said bacterial strain is spray dried. In certain embodiments, the invention provides the above pharmaceutical composition, wherein the bacterial strain is lyophilized or spray dried and wherein it is live. In certain embodiments, the invention provides the above pharmaceutical composition, wherein the bacterial strain is lyophilized or spray dried and wherein it is viable. In certain embodiments, the invention provides the above pharmaceutical composition, wherein the bacterial strain is lyophilized or spray dried and wherein it is capable of partially or totally colonizing the intestine. In certain embodiments, the invention provides the above pharmaceutical composition, wherein the bacterial strain is lyophilized or spray dried and wherein it is viable and capable of partially or totally colonizing the intestine.

### Culturing Methods

The bacterial strains for use in the present invention can be cultured using standard microbiology techniques as detailed in, for example, Handbook of Microbiological Media, Fourth Edition (2010) Ronald Atlas, CRC Press; Maintaining Cultures for Biotechnology and Industry (1996) Jennie C. Hunter-Cevera, Academic Press; Strobel (2009) Methods Mol Biol. 581:247-61.

### Therapeutical Use

One or more Lachnospiraceae spp and/or Ruminococcus lactaris strains of the invention may be for use alone or in combination in the treatment and/or prevention of a neuropsychiatric disease or disorder causing memory deficits. In one embodiment, the Lachnospiraceae spp and/or Ruminococcus lactaris strains of the invention may be for use in the treatment or prevention of Alzheimer's disease or aging-related memory deficits such as vascular dementia. In some embodiments, the memory deficit is caused by agnosia, amnesia, traumatic brain injury, dementia, postoperative cognitive dysfunction, Attention Deficit/Hyperactivity Disorder, Parkinson disease, Huntington disease, or Amyotrophic Lateral Sclerosis. In some embodiments, the memory deficit is caused by agnosia, amnesia, traumatic brain injury, dementia, postoperative cognitive dysfunction, Attention Deficit/Hyperactivity Disorder. In some embodiments, the memory deficit is short-term memory loss. In some embodiments, the memory deficit is long-term memory loss. In some embodiments, the Alzheimer's Disease is due to genetic causes. In some embodiments, the compositions of the invention may be useful as nootropic agents (e.g., memory enhancers).

As used herein, the term “memory” refers to acquired information stored in the brain that can be retrieved, or the ability to encode, store, register, access, and/or retrieve the acquired information. In some embodiments, the terms “memory” and “memory ability” are used interchangeably in the present invention to refer to the ability to encode, store, register, access, and/or retrieve information. In some embodiments, the memory may include a short-term memory and/or a long-term memory. In some embodiments, the short-term memory may refer to a capability for holding a small amount of information in the brain for a short period of time (e.g., 1s, 2s, 3s, 4s, 5s, 10s, 15s, 20s, 25s, 30s, or the like). In some embodiments, the short-term memory may be formed rapidly and may last for a relatively short period of time (e.g., one or more seconds, one or more minutes, one or more hours, or one or more days, or the like). In some embodiments, the long-term memory may be a stage of the Atkinson-Shiffrin memory model in which informative knowledge may be held indefinitely. In some embodiments, the long-term memory may be formed less rapidly and may last for a relatively long period of time (e.g., one or more days, one or more weeks, one or more months, or one or more years, or the like) in comparison with the short-term term memory. In some embodiments, newly acquired information may be initially stored in the brain in a fragile state and may tend to be gradually forgotten by the subject. In a memory consolidation process, the fragile state of the acquired information may be transformed into a relatively stable state in the brain, and accordingly, the acquired information is less likely to be forgotten by the subject. In some embodiments, the memory consolidation process may occur naturally over time or with the re-acquisition of the same acquired information (or related information).

As used herein the term “memory deficit” may refer to a symptom that the subject abnormally has difficulty in encoding, storing, registering, accessing, and/or retrieving acquired information. In one embodiment, treatment or prevention of memory deficits can be measured as suggested in the models used in the Examples. Clinical and animal studies have indicated that the most vulnerable cognitive domains, explicit memory and spatial memory, are dependent on the hippocampus. Consequently, methods that assess the activity and/or neural functions of the subject's hippocampus may be used to assess memory deficits. In some embodiments, the memory deficit is assessed with an object recognition task. In some embodiments, verbal short-term memory is tested by digit span tasks in which the individual is exposed to numbers containing various digits for various times and asked to recall the digits sometime later. In one embodiment, nonverbal short-term memory may be tested by various motor or spatial memory tasks, such as spatial information tests. In these tasks, the subject is exposed to various motor tasks or spatial orientations and asked to recall or reconstruct them later. Procedural memory is that memory underlying motor performance or skills. In humans, the separation of procedural and declarative memory is not a simple task, because the human may develop declarative memory strategies for motor performances. However, this type of memory can be tested with various tasks such as mirror drawing tests, mirror reading tasks, weight sampling tasks, speed reading of repeated nonwords, and resolving random-dot stereograms. In addition, various forms of classical conditioning tasks, such as eyeblink conditioning in which a tone or light onset is paired with an air puff to the eye can be used to test procedural memory function. This type of task has many forms and variations which have been used on various deficits and conditions. Many other tests of human procedural memory exist, such as those used to test classification abilities which are included in procedural memory. Here, subjects are tested on ability to classify letter strings as grammatical or nongrammatical, among other tests. Declarative memory may be tested with many sorts of tests, including fact recall, matching tests of various sorts, tests for retention from minutes to days or years, verbal learning and recall tasks, and many other such tests. Here the number and variety of tests is very great and depend on whether the deficit is thought to be in autobiographical (episodic) or world facts (semantic) memory systems.

In one preferred embodiment, the memory deficit is thus assessed with one or more object recognition tasks; digit span tasks; motor or spatial memory tasks, such as spatial information tests; mirror drawing tests; mirror reading tasks; weight sampling tasks; speed reading of repeated nonwords; resolving random-dot stereograms; classical conditioning tasks, such as eyeblink conditioning; ability to classify letter strings as grammatical or nongrammatical; fact recall; matching tests of various sorts; tests for retention from minutes to days or years; verbal learning and recall tasks, and combinations of the same.

In one embodiment, the subject is identified for treatment based on the diagnosis of Alzheimer's disease or risk for Alzheimer's disease. In another embodiment, the subject is identified for treatment based on their age. In another embodiment, the subject is identified as having symptoms of deficit in memory ability (assessed by, in one embodiment, the methods of the previous paragraph), another neuropsychological test result, a magnetoencephalography (MEG) testing result, a brain imaging assessment (e.g., Computed Tomography (CT), Magnetic Resonance Imaging (MRI), or Positron Emission Tomography (PET)) result, or the like, or any combination thereof.

The bacteria or bacterial compositions may be provided once or twice; chronically, in a continuous mode for a certain period of time; or intermittently, with interruptions or in cycles. Combinations of bacteria may be administered simultaneously (e.g., as part of the same composition), or separately, e.g. successively. Typically, the bacteria or bacterial composition is administered in an effective amount, such as e.g., an amount sufficient to colonize the gastrointestinal tract of a subject, for a suitable period of time. In one embodiment, an effective amount includes a therapeutically effective amount or a prophylactically effective amount. A “therapeutically effective amount” refers to an amount effective, at dosages and for periods of time necessary, to achieve the desired therapeutic result, such reduction in the memory deficits as assessed in the previous paragraph. A therapeutically effective amount of a (bacterial) composition may vary according to factors such as the disease state, age, sex, and weight of the subject, and the ability of the (bacterial) composition to elicit a desired response in the individual. Dosage regimens may be adjusted to provide the optimum therapeutic response. A “prophylactically effective amount” refers to an amount effective, at dosages and for periods of time necessary, to achieve the desired prophylactic result. Typically, a prophylactic dose is used in subjects prior to or at an earlier stage of disease or disease symptoms, so that a prophylactically effective amount may be less than a therapeutically effective amount. In some embodiments, the composition is administered to subjects at risk for memory deficits. “At risk” subjects are those who are more susceptible to developing memory deficits in comparison to a member of the general public.

A suitable range for therapeutically or prophylactically effective amounts, or probiotic amounts, of bacteria or a bacterial composition, as described herein, will be determined by the skilled person, and may include without limitation at least or about 10°, 10¹, 10², 10³, 10⁴, 10⁵, 10⁶, 10⁷, 10⁸, 10⁹, 10¹⁰, 10¹¹, 10¹², 10¹³, or 10¹⁴ colony forming units (CFU) of the bacteria, per unit dosage, in particular between 102 and 1010 CFUs per unit dose (the amount of a medication administered to a patient in a single dose). In some embodiments, dosages for live bacteria, in vegetative or spore forms, can be about 0.1 to about 1000 mg, such as about 0.5 mg to about 5 mg, about 1 mg to about 1000 mg, about 2 mg to about 200 mg, about 2 mg to about 100 mg, about 2 mg to about 50 mg, about 4 mg to about 25 mg, about 5 mg to about 20 mg, about 10 mg to about 15 mg, about 50 mg to about 200 mg, about 200 mg to about 1000 mg, or about 1, 2, 3, 4, 5 or more than g per dose or composition; or 0.001 mg to 1 mg, 0.5 mg to 5 mg, 1 mg to 1000 mg, 2 mg to 200 mg, or 2 mg to 100 mg, or 2 mg to 50 mg, or 4 mg to 25 mg, or 5 mg to 20 mg, or 10 mg to 15 mg, or 50 mg to 200 mg, or 200 mg to 1000 mg, or 1, 2, 3, 4, 5 or more than 5 g per dose or composition. Dosage values may vary with the severity of the condition to be alleviated. For any particular subject, specific dosage regimens may be adjusted over time according to the individual need and the professional judgement of the person administering or supervising the administration of the compositions. For example, a single bolus may be administered, several divided doses may be administered over time or the dose may be proportionally reduced or increased as indicated by the exigencies of the situation. The bacteria or composition of the invention may be administered daily or more frequently, such as twice or more per day. “Probiotic” refers to live microbes that, when consumed at adequate amounts, provide a health benefit to the host. Often these microbes are similar to microbes resident in the human gut, but may be found in very small numbers (e.g., as evidenced by dysbiosis), or they may require to process prebiotics or other substrates to encourage growth of other microbes.

In one embodiment, the suitable daily dose of a bacterial strain according to the invention is from 10⁷ to 10¹² viable cells per ml (vc/ml), more preferably from 10⁹ to 10¹⁰ vc/ml as a medicament, for example as a daily dose equivalent to 10⁹ vc/ml.

In one embodiment, the strains of the invention are live biotherapeutic products (LBP) whose activity lies in the gut. An LBP bacterium according to the invention denotes a bacterium which ingested live in adequate quantities can exert beneficial effects on the human health. In a preferred embodiment, these strains are administered alive to the gut. The bacteria strains of the invention may be administered to the gut of an individual to be treated by different ways, i.e., by the oral, rectal or parenteral route. A bacterium according to the invention is preferably administered by the oral or rectal route, more preferably by the oral route.

In one embodiment, the strains of the invention may be used as feed additives. The feed additive may be administered alone or in combination with other feed additives in the edible carrier. In addition, the feed additive may be used in the animal as a coating, directly mixed with the animal feed, or as a separate oral formulation. When the feed additive is administered separately from the animal feed, it is possible to combine it with a food-to-be acceptable edible carrier known in the art, and can be prepared immediately in an immediate or a sustained release formulation. Such edible carriers may be solid or liquid, such as corn starch, lactose, sucrose, peanut oil, olive oil, sesame oil and propylene glycol. The feed or feed additive of the present invention may be administered to a variety of animals including mammals (e.g., pet dogs), poultry, and fish.

In one embodiment, the strains of the invention are used in combination with other therapeutic agents to treat the disease or disorder underlying the memory deficit or to treat other diseases. In some embodiments, the strains of the invention are used simultaneously. In some embodiments, the strains of the invention and the other therapeutic agent(s) are used sequentially (before and/or after). In some embodiments, one or more of the therapeutic agents are added to the compositions comprising the bacteria of the invention. In some embodiments, the bacteria of the invention are used in combination with other bacteria. They may be used as separate compositions in combination or used in the same composition. As used herein, “simultaneous” means that the therapeutic agent and the composition of the invention are to be or have been administered at the same time or as part of the same treatment regimen; “sequentially” means that doses of the therapeutic agent and the composition of the invention are to be or have been administered concurrently or as part of the same treatment regimen; and “separately” means that the complete dosage of the therapeutic agent and the composition of the invention are to be or have been administered one after the other as part of the same treatment regimen. In the context of these embodiments, “treatment regimen” refers to the prescription of a treatment program comprising the administration of both a therapeutic agent and the composition of the invention to a subject, i.e., where a physician actively prescribes both of the therapeutic agent and the composition of the invention at the same time for the treatment or prevention of memory deficit or a disease, disorder, or condition causing memory deficit.

In one embodiment, the bacteria of the invention are administered together with other therapeutic agents used in the treatment of the disorder underlying the memory deficits. In one embodiment, the therapeutic agent(s) is used in the prophylaxis or treatment of Alzheimer's disease. In one embodiment, the bacteria of the invention are administered together with other therapeutic agents use to “delay” aging. In one embodiment, such treatment is calorie restriction. Thus, the invention encompasses the use of the compositions of the invention for the treatment or prevention of Alzheimer's disease or aging-related memory deficits.

In one embodiment, pharmacological therapeutic treatments for Alzheimer's disease can be divided into two categories: symptomatic treatments such as acetylcholinesterase inhibitors and N-methyl-D-aspartate (NMDA) receptor antagonists and etiology-based treatments such as secretase inhibitors, amyloid binders, and tau therapies. In one embodiment, strategies of for prevention Alzheimer's disease through nonpharmacological treatments are caused by lifestyle interventions such as exercise, mental challenges, and socialization as well as caloric restriction and a healthy diet. In one embodiment, the bacteria of the invention are used in combination with any of the listed examples of therapies of Alzheimer's disease. In some embodiments, the therapeutic agents and the bacteria are in the same composition.

### Therapeutic Method

The present invention also relates to a method of treatment and/or prevention of memory deficits in a subject in need thereof by administering a composition comprising bacteria of one or more strains selected from Lachnospiraceae spp and Ruminococcus lactaris strains to the subject.

In one embodiment, the memory deficit is due to aging.

In one embodiment, the memory deficit is caused by a neuropsychiatric and/or neurodegenerative disease, disorder, or condition. Preferably, said neuropsychiatric and/or neurodegenerative disease, disorder, or condition is selected from Alzheimer's disease, agnosia, amnesia, traumatic brain injury, dementia, postoperative cognitive dysfunction, Attention Deficit/Hyperactivity Disorder, Parkinson disease, Huntington disease or Amyotrophic Lateral Sclerosis. More preferably, the neuropsychiatric and/or neurodegenerative disease, disorder, or condition is selected from Alzheimer's disease, agnosia, amnesia, traumatic brain injury, dementia, postoperative cognitive dysfunction, or Attention Deficit/Hyperactivity Disorder.

The invention also provides a method of treating or preventing Alzheimer's disease in a subject in need thereof, by administering a composition comprising bacteria of one or more strains selected from Lachnospiraceae spp and Ruminococcus lactaris strains, preferably from Ruminococcus lactaris strains, to the subject.

The invention also provides a method of treating or preventing Alzheimer's disease at different stages of the disease, in a subject in need thereof, by administering a composition comprising bacteria of one or more strains selected from Lachnospiraceae spp and Ruminococcus lactaris strains, preferably from Ruminococcus lactaris strains, to the subject.

In one embodiment, the invention also provides a method of treating or preventing Alzheimer's disease at the preclinical stage, in a subject in need thereof, by administering a composition comprising bacteria of one or more strains selected from Lachnospiraceae spp and Ruminococcus lactaris strains, preferably from Ruminococcus lactaris strains, to the subject.

In one embodiment, the invention also provides a method of treating or preventing Alzheimer's disease at the mild cognitive impairment stage in a subject in need thereof, by administering a composition comprising bacteria of one or more strains selected from Lachnospiraceae spp and Ruminococcus lactaris strains, preferably from Ruminococcus lactaris strains, to the subject.

In one embodiment, the invention also provides a method of treating or preventing Alzheimer's disease at the mild dementia stage in a subject in need thereof, by administering a composition comprising bacteria of one or more strains selected from Lachnospiraceae spp and Ruminococcus lactaris strains, preferably from Ruminococcus lactaris strains, to the subject.

In one embodiment, the invention also provides a method of treating or preventing Alzheimer's disease at the moderate dementia stage, in a subject in need thereof, by administering a composition comprising bacteria of one or more strains selected from Lachnospiraceae spp and Ruminococcus lactaris strains, preferably from Ruminococcus lactaris strains, to the subject.

In one embodiment, the invention also provides a method of treating or preventing Alzheimer's disease at the severe dementia stage, in a subject in need thereof, by administering a composition comprising bacteria of one or more strains selected from Lachnospiraceae spp and Ruminococcus lactaris strains, preferably from Ruminococcus lactaris strains, to the subject.

Preferably, the subject is a warm-blooded animal, more preferably a human.

In one embodiment, the bacteria are of any one of the strains deposited to the CNCM under the accession number CNCM I-5830 and CNCM I-5831, respectively.

In one embodiment, the bacteria are comprised in a physiologically acceptable composition.

In one embodiment, this composition further comprises a prebiotic.

In one embodiment, this composition further comprises a therapeutic agent.

In one embodiment, this composition further comprises another compound so as the composition is formulated for use as food.

### Mouse Model of the Invention

In a further aspect, the present invention provides a new mouse model called “human Alzheimer's disease-like” or “human model of Alzheimer's disease”. This mouse hosts a gut microbiota of human Alzheimer's disease-affected patients. It has been obtained by transferring the gut microbiota obtained from fecal samples of Alzheimer's disease-affected patients following the protocol described in Rei et al, 2021 in RjOrl: SWISS mice, preferably in male RjOrl: SWISS mice.

Prior to the FMT, the mice are preferably treated with a protocol of bowel cleansing, which consists in administering broad-spectrum antibiotics as used in Rei et al, 2021 or in the oral administration of a laxative solution, preferably in the oral administration of a laxative solution.

In one embodiment, the laxative solution comprises polyethylene glycol. In one embodiment the laxative solution comprises between 50 and 90% by weight of polyethylene glycol. In one preferred embodiment the laxative solution comprises between 60 and 80% by weight of polyethylene glycol.

In one embodiment, the polyethylene glycol is PEG 3350.

In one embodiment, the laxative solution is a COLOPEG solution in particular COLOPEG solution Macrogol 3350 sold by LABORATOIRES BOUCHARA-RECORDATI.

In one embodiment, the laxative solution is administered by force-feeding cannula to the mouse.

In one embodiment, the laxative solution is administered preferably at least twice, more preferably at least three times and even more preferably five times.

In one embodiment, the laxative solution is administered preferably at least twice with a 30 minutes interval, more preferably at least three times with a 30 minutes interval and even more preferably five times with a 30 minutes interval.

In one embodiment, the laxative solution is administered in a volume of between 100 and 500 μl preferably, between 200 and 300 μl per administration.

In one embodiment, before the administration of the laxative solution, the mouse is fasted at least one hour, preferable about two hours.

In one embodiment, the fecal samples of AD-affected patients are suspended in an aqueous solution before injection to the mouse.

In one embodiment, the feces are diluted to a ratio of between 1/40 and 1/10 in volume in the aqueous solution and preferably about 1/20 in volume in the aqueous solution.

In one embodiment, the aqueous solution of feces is administered by force-feeding cannula to the mouse.

In one embodiment, the aqueous solution of feces is administered in a volume of between 100 and 500 μl preferably, between 150 and 300 μl per administration.

In one embodiment, the aqueous solution of feces is administered preferably at least twice, more preferably at least three times.

In one embodiment, the aqueous solution of feces is administered preferably at least twice with a one-day interval, more preferably at least three times with one-day interval.

The animals of the thus obtained mice model experience memory loss that is typical of Alzheimer's disease symptoms about 28 days after the FMT. As shown in example 2 below, the animals experience memory deficits in the isotropic version of the novel object location (ISO-NOL), isotropic version of the novel object recognition (ISO-NOR) and fear conditioning tasks, and they display downregulation of synaptic plasticity genes and upregulation of neuroinflammation-associated markers.

The present invention thus further provides a method of generating a mouse model exhibiting a neuropsychiatric disease and/or a neurodegenerative disease's symptoms, said method comprising the step of transferring gut microbiota obtained from fecal samples of the neuropsychiatric disease and/or a neurodegenerative disease-affected human patients to the mouse.

In one embodiment, the said neuropsychiatric disease and/or a neurodegenerative disease is selected from Alzheimer's disease, Parkinson disease, Huntington disease or Amyotrophic Lateral Sclerosis.

The present invention thus further provides a method of generating a mouse model exhibiting Alzheimer's disease symptoms, said method comprising the step of transferring gut microbiota obtained from fecal samples of Alzheimer-affected human patients to the mouse.

In one embodiment, the method of the invention further comprises, before the step of transferring gut microbiota obtained from fecal samples of Alzheimer-affected human patients, a step of performing a bowel cleansing to the mouse, preferably by administering broad-spectrum antibiotics or by oral administration of a laxative solution.

The present invention thus also further provides a mouse model produced by the above-mentioned methods.

### EXAMPLES

### Example 1: Sorting and Cultivation of Bacterial Strains from Healthy Young Donor Fecal Samples and Phylogenic Characterization of Lachnospiraceae Spp Cncm I-5830 and Ruminococcus Lactaris Cncm I-5831

In order to obtain bacterial strains to be tested for their anti-Alzheimer's disease and aging effect, a method of species-targeted sorting and cultivation of gut microbiota bacterial strains from healthy young human donor fecal samples through flow cytometry under anaerobic conditions was used. Antibodies used were directed against the Faecalibacterium praunitzii (ATCC 27766 plus ATCC 27768) and Roseburia intestinalis (DSM 14610) strains. As this method allows the sorting of bacterial strains of the targeted species but also of others, it was used as a mean to constitute a collection of young adult human donor bacterial strains to be tested for their anti-Alzheimer's disease/aging potential. This led to the collection of a large number of individual bacterial strains of a variety of different families. Cultivable candidates were then independently grown and 16S sequencing on their extracted DNA allowed their phylogenic identification.

(a) Species-Targeted Sorting of Gut Microbiota Bacterial Strains from Healthy Young Human Donor Fecal Samples

Fecal samples were collected from healthy young (18 to 40 years old) donors, exempt of any pathology, and/or any drug prescription, with the absence of any antibiotic use in the previous three months prior to the collect. Table 1 highlights the age and sex of the donors used for the fecal sample collection.

TABLE 1
Information on fecal samples donor
used to sort the bacterial strains

| Donor ID | Age | Sex |
|---|---|---|
| 1006-B-176 | 20 years | male |
| 1006-M-1-013 | 20 years | male |
| 1006-B-1-185 | 19 years | Female |
| 1006-B-1-182 | 36 years | Female |
| 1006-B-1-184 | 20 years | Female |

Fecal samples were collected on site and immediately processed. For each sample, 1g of fecal material was collected in collection tubes. All subsequent steps were performed in an anaerobic chamber using sterile reduced liquid media. The Faecalicatena contorta, Lachnospiraceae spp and Ruminococcus lactaris strains were isolated using a protocol identical to that described in Bellais, S. et al., 2020 (Bellais et al., 2020). In brief, for each sample, 1 g of fecal material was suspended in 10 ml PBS and homogenized, filtered through a 70-μm cell strainer. LIVE/DEAD™ staining was used to select live bacteria, whereas polyclonal antibodies were used to enrich with the target bacterial species. Staining was performed in anaerobic conditions for 30 min in the dark, and bacteria were then washed in reduced PBS before analysis. Bacteria were gated based on forward scatter (FSC) and side scatter (SSC) parameters. Live ones were selected according to SYTO 9/PI fluorescence and events collected from the antibodies-stained gates were sorted on mGAM-CRI plates. Plates were then incubated for 5 days at 37° C. in anaerobic conditions.

The sorting led to the collection of a large number of bacterial strains. A portion were Faecalibacterium praunitzii and Roseburia intestinalis strains, but the rest belonged to other families. This was probably due to the non-exclusive nature of the epitopes recognized by the antibodies, and to some non-specific binding. Table 2 depicts the donor and flow cytometry information for the isolation of the 1006-B-1-182_18-4, 1006-B-1-182_21-1 (CNCM I-5831) and 1006-M-1-013_15-5 (CNCM I-5830) strains.

TABLE 2
sorting related information of the strains and their phylogenic classification

| Donor | Date | Antibody used | Sorted | CNCM | |
|---|---|---|---|---|---|
| ID | sorting | for sorting | strain-code | ID | Classification |
| 1006-B- | 30 Nov. 2020 | anti F. praunitzii | 18-4 | — | Faecalicatena |
| 1-182 | | ATCC 27766/27768 | | | contorta |
| 1006-B- | 30 Nov. 2020 | anti R. intestinalis | 21-1 | I-5830 | Lachnospiraceae |
| 1-182 | | DSM 14610 | | | spp |
| 1006-M- | 30 Nov. 2020 | anti R. intestinalis | 15-5 | I-5831 | Ruminococcus |
| 1-013 | | DSM 14610 | | | lactaris |

### (B) DNA Extraction and Cryoprotection of Faecalicatena Contorta 1006-B-1-182 18-4. Lachnospiraceae Spp CNCM I-5830 and Ruminococcus Lactaris CNCM I-5831 Strains

DNA extraction and bacteria cryoprotection was made on the 1006-B-1-182_18-4, 1006-M-1-013_15-5 (CNCM I-5830) and 1006-B-1-182_21-1 (CNCM I-5831) strains, to allow for their subsequent 16S DNA sequencing and culture, respectively.

Colonies of the sorted 1006-B-1-182_18-4, 1006-M-1-013_15-5 (CNCM I-5830) and 1006-B-1-182_21-1 (CNCM I-5831) strains were visually selected on the mGAM-CRI sorting plates and subcultivated on mGAM plates. DNA samples were extracted from isolated colonies of the different strains by collection of 2 to 4 strain colonies in Instagen matrix (Biorad) and following “DNA preparation for PCR” instruction of the provider. A similar number of colonies were also collected in reduced 16% glycerol in PBS and stored at −80° C. for cryoprotection of the strains and their subsequent cultures.

Strains 013-HF_15-5 and 182-OH_21-1 was deposited to the “Collection Nationale de Cultures de Micro-organismes” (CNCM) micro-organism bank under the reference CNCM 1-5830 and CNCM I-5831, respectively. Table 2 above shows the correspondence between the flow cytometry related information and the CNCM identification code for the strains CNCM I-5830 and CNCM I-5831.

### (C) Phylogenic Characterization of Lachnospiraceae Spp CNCM I-5830 and Ruminococcus Lactaris CNCM I-5831 Strains

Lachnospiraceae spp and Ruminococcus lactaris 16S rRNA sequences were analyzed.

DNA samples from the Faecalicatena contorta 1006-B-1-182_18-4, Lachnospiraceae spp CNCM I-5830 and Ruminococcus lactaris CNCM I-5831 strains were used to perform PCR amplification of the 16S rRNA gene using 27F-YM and 1391R primer set. The result products were sent for sequencing to Eurofins (Cologne, Germany).

From the result sequences, pairwise sequence similarities were calculated using the method recommended by Meier-Kolthoff et al. (2013) for the 16S rRNA gene available via the GGDC web server. Phylogenies were inferred by the GGDC web server (Meier-Kolthoff et al. 2022) available at https://urldefense.com/v3/_http://ggdc.dsmz.de/__;!! JFdNOqOXpB6UZW0!-aXbUPgCyD4K36hsYEddOSdpKKIRdOy_uYRXwty43D1t06alsnxh1 HrZblc3mvcMKQ$ using the DSMZ phylogenomic pipeline (Meier-Kolthoff et al. 2014) adapted to single genes.

A multiple sequence alignment was created with MUSCLE (Edgar 2004, Nucleic Acids Res 32:1792-1797). Maximum likelihood (ML) and maximum parsimony (MP) trees were inferred from the alignment with RaxML (Stamatakis 2014, Bioinformatics 30:1312-1313) and TNT (Goloboff et al. 2008, Cladistics 24:774-786), respectively. For ML, rapid bootstrapping in conjunction with the autoMRE bootstopping criterion (Pattengale et al. 2010, J Comput Biol 17:337-354) and subsequent search for the best tree was used; for MP, 1000 bootstrapping replicates were used in conjunction with tree-bisection-and-reconnection branch swapping and ten random sequence addition replicates. The sequences were checked for a compositional bias using the X2 test as implemented in PAUP* (Swofford 2002, version 4.0 b10. Sinauer Associates, Sunderland).

Full-length 16S rRNA sequencing of the isolated bacterial strains were determined.

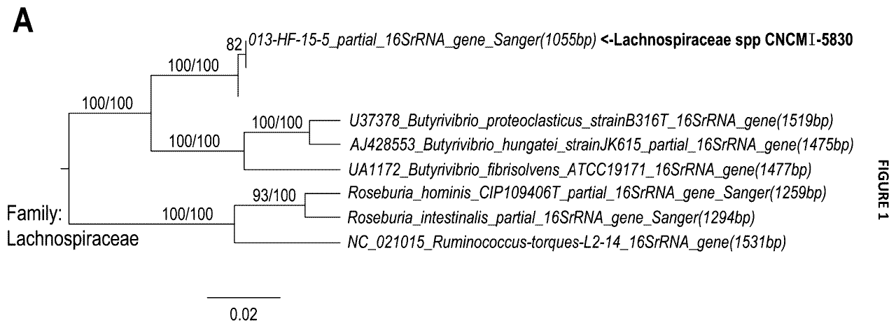

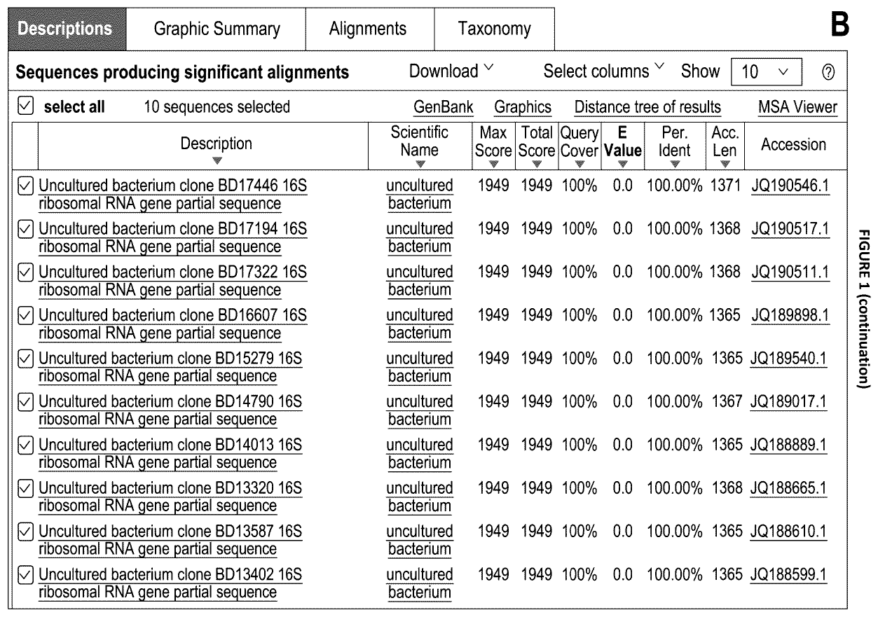

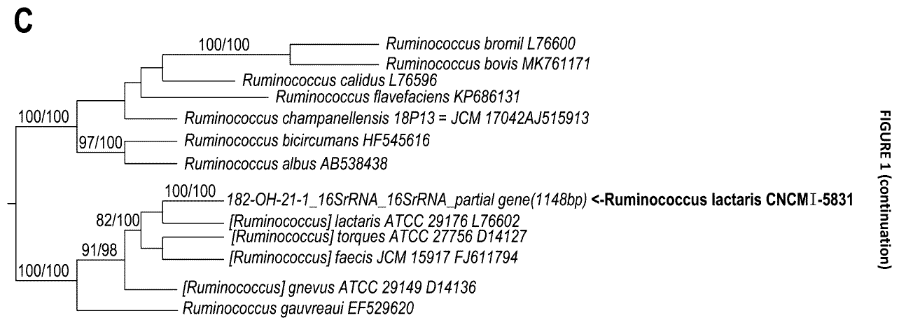

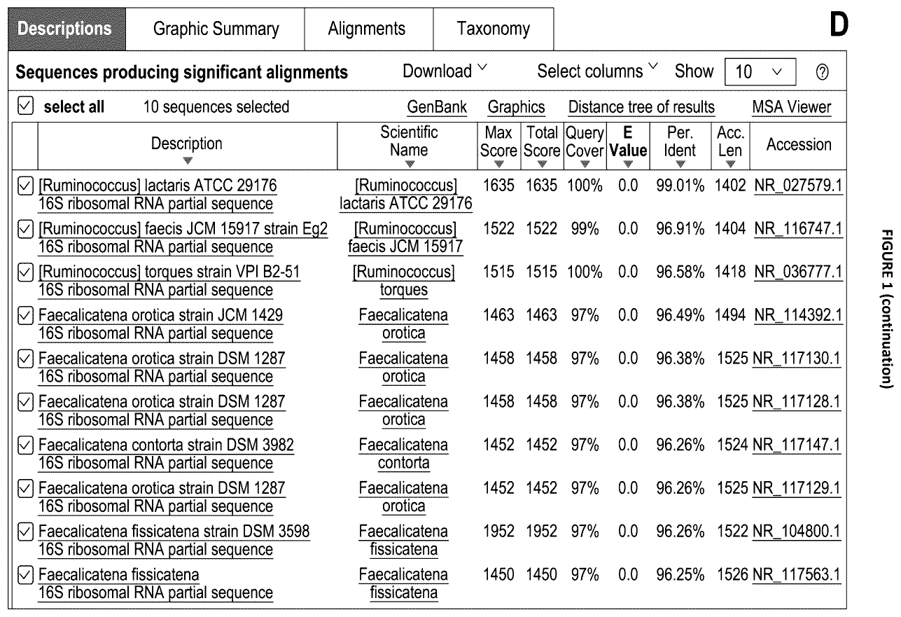

DSMZ phylogenic pipeline analysis on the strain 182-0H_18-4 16S rRNA gene sequence allowed its classification as a Faecalicatena contorta (data not shown). A similar analysis on the 16S rRNA gene sequence of strain 013-HF_15-5 CNCM I-5830 (SEQ ID NO: 1), showed it to be a new strain and phylogenetically the closest to a group of uncharacterized strains (FIG. 1A). The closest known bacterial strains are Butyrivibrio proteoclasticus and Butyrivibrio hungatei. It was therefore named based on the closest known ascending phylogeny, i.e., the family order: Lachnospiraceae. This strain was therefore classified as a Lachnospiraceae spp. Results from performing a nucleotide BLAST (https://blast.ncbi.nlm.nih.gov/Blast.cgi) on the 013-HF_15-5 CNCM I-5830 16S rRNA gene sequence, showed the 10 bacterial species with the highest proximity to the 16S rRNA gene sequence of strain CNCM I-5830 to be all uncharacterized bacterial strains (FIG. 1B).

As of the 16S rRNA gene sequence of the 182-OH_21-1 CNCM I-5831 strain (SEQ ID NO: 2), DSMZ phylogenic analysis showed it to be a new strain belonging to the phylogenic branch of Ruminococcus lactaris strains (FIG. 1C). Nucleotide BLAST analysis of the 16S rRNA gene sequence of this strain, showed it to be different from other members of the genus with a closest sequence analogy of 99,01% with Ruminococcus lactaris strain ATCC 29176 (FIG. 1D).

SEQ ID NO: 1: “013-HF_15-5”: partial 16S rRNA gene sequence (1055 bp)
of Lachnospiraceae spp CNCM I-5830
TTATAAACTTAGTGGCGGACGGGTGAGTAACACGTGGAGAACCTGCCTCTTACC
GGGGGACAGCAGTTGGAAACGACTGATAATACCGCATAAGCGCACGGGACCGC
ATGGTCAAGTGTGAAAAGATTTATCGGTAAGAGATGGCTCCGCGTCTGATTAGC
CAGTTGGCGGGGTAACGGCCCACCAAAGCGACGATCAGTAGCCGGCCTGAGA
GGGTGAACGGCCACATTGGGACTGAGACACGGCCCAGACTCCTACGGGAGGC
AGCAGTGGGGGATATTGCACAATGGAGGAAACTCTGATGCAGCGACGCCGCGT
GAGTGAAGAAGTATTTCGGTATGTAAAGCTCTATCAGCAGGGAAGAAAGACCTC
GAAAGAGGGGATGACGGTACCTGAGTAAGAAGCCCCGGCTAACTACGTGCCAG
CAGCCGCGGTAATACGTAGGGGGCGAGCGTTATCCGGATTTACTGGGTGTAAA
GGGAGCGCAGACGGAAGAGCAAGTCTGATGTGAAAACCCGGGGCTCAACCCC
GGGCCTGCATTGGAAACTGTTTTTCTGGAGTACTGGAAGGGCAGGCGGAATTC
CTGGTGTAGCGGTGAAATGCTTAGATATCAGGAGGAACACCGGTGGCGAAGGC
GGCCTGCTGGACAGTAACTGACGTTGAGGCTCGAAAGCGTGGGGAGCAAACAG
GATTAGATACCCTGGTAGTCCACGCCGTAAACGATGAATACTAGGTGTTGGAAG
GCAAAGCCTTTCAGTGCCGTCGCAAACGCATTAAGTATTCCACCTGGGGAGTAC
GTTCGCAAGAATGAAACTCAAAGGAATTGACGGGGACCCGCACAAGCGGTGGA
GCATGTGGTTTAATTCGAAGCAACGCGAAGAACCTTACCAGACCTTGACATCCC
TCTGAATACGGTGTAATGACTGTAGGCCTTCGGGACAGAGGAGACAGGTGGTG
CATGGTTGTCGTCAGCTCGTGTCGTGAGATGTTGGGTCAAGTCCCGCAACGAG
CGCAACCCTTGTCCATAGTAGCCAGCAGTAAGATGGGAACTCTAT
SEQ ID NO: 2: “182-OH_21-1 strain”: partial 16SrRNA gene sequence
(1238 bp) of Ruminococcus lactaris CNCM I-5831
ACTGAGCGGCGGACGGGTGAGTAACGCGTGGGTAACCTGCCTCATACAGGGG
GATAACAGTTAGAAATGACTGCTAATACCGCATAAGACCACGGTACCGCATGGT
ACAGTGGGAAAAACTCCGGTGGTATGAGATGGACCCGCGTCTGATTAGCTAGTT
GGTAAGGTAACGGCTTACCAAGGCGACGATCAGTAGCCGACCTGAGAGGGTGA
CCGGCCACATTGGGACTGAGACACGGCCCAAACTCCTACGGGAGGCAGCAGT
GGGGAATATTGCACAATGGGGGAAACCCTGATGCAGCGACGCCGCGTGAAGG
ATGAAGTATTTCGGTATGTAAACTTCTATCAGCAGGGAAGAAAATGACGGTACCT
GACTAAGAAGCCCCGGCTAACTACGTGCCAGCAGCCGCGGTAATACGTAGGGG
GCAAGCGTTATCCGGATTTACTGGGTGTAAAGGGAGCGTAGACGGTTATGTAAG
TCTGATGTGAAAACCCGGGGCTCAACCCCGGGACTGCATTGGAAACTATGTAAC
TAGAGTGTCGGAGAGGTAAGTGGAATTCCTAGTGAGCGGTGAAATGCGTAGAT
ATTAGGAGGAACACCAGTGGCGAAGGCGGCTTACTGGACGATGACTGACGTTG
AGGCTCGAAAGCGTGGGGAGCAAACAGGATTAGATACCCTGGTAGTCCACGCC
GTAAACGATGCATACTAGGTGTCGGGTGGCAAAGCCATTCGGTGCCGCAGCAA
ACGCAATAAGTATGCCACCTGGGGAGTACGTTCGCAAGAATGAAACTCAAAGGA
ATTGACGGGGACCCGCACAAGCGGTGGAGCATGTGGTTTAATTCGAAGCAACG
CGAAGAACCTTACCTGCTCTTGACATCCCCCTGACCGGCGCGTAATGGTGCCTT
TCCTTCGGGACAGGGGAGACAGGTGGTGCATGGTTGTCGTCAGCTCGTGTCGT
GAGATGTTGGGTTAAGTCCCGCAACGAGCGCAACCCTTATCTTTAGTAGCCAGC
GGTTTGGCCGGGCACTCTAGAGAGACTGCCAGGGATAACCTGGAGGAAGGTG
GGGATGACGTCAAATCATCATGCCCCTTATGAGCAGGGCTACACACGTGCTACA
ATGGCGTAAACAAAGGGAGGCGAAGCCGCGAGGTGGAGCAAATCCCAAAAATA
ACGTCTCAGTTCGGATTGTAGTCTGCAACTCGACTACATGAAGCTGGAATCGCT
AGTAATCGCGA

### Example 2: Effect of Lachnospiraceae Spp CNCM I-5830 or Ruminococcus Lactaris CNCM 1-5831 on Memory Abilities in Mouse Models of Alzheimer's Disease and Aging

Faecalicatena contorta, Lachnospiraceae spp and Ruminococcus lactaris strains according to the invention were tested for their ability to have a direct impact on Alzheimer's disease-related memory deficits in a mouse model of Alzheimer's disease.

(a) Mice Transplanted with the GM from Alzheimer's Disease-Affected Human Donors (Human Alzheimer's Disease-Like Mice)

Mice were FM transplanted with the GM from either young human subjects or A Alzheimer's disease D-affected patients. The recruitment of Alzheimer's disease-affected donors was made at the Sainte Perine hospital (APHP, Paris, France). Inclusion criteria included being diagnosed with Alzheimer's disease on the basis of clinical examination and a score below 24 on the Mini-Mental State Exam (MMSE). Exclusion criteria included absence of other forms of dementia (notably vascular dementia), and of any antibiotic use in the previous month prior to the collect and being devoid of any pathology and/or any drug prescription. Fecal samples collection from young (18 to 35 years old) were selected from the Institut Pasteur (IP) ICAReB plateform's healthy volunteer Diagmicoll and CoSImmGEn cohorts. GM from both young and Alzheimer's disease-affected donors was made following protocol described in (Rei et al., 2021). Collection was made at the Sainte Perine hospital for Alzheimer's disease-affected subjects and at home for young control donors.

Experiments were performed using adult (10 to 12-week-old) male RjOrl: SWISS mice purchased from Janvier labs (St Berthevin, France). Animals prior to human FMT were treated with a protocol of bowel cleansing followed by FMT according to the protocol from (Rei et al., 2021).

The strains treatment (described below) to assess their effect on memory abilities in the human GM model of Alzheimer's disease was started 28d post-FMT, a timing where mice were previously shown to phenocopy the memory loss seen in Alzheimer's disease, with memory deficits in the isotropic version of the novel object location (ISO-NOL), isotropic version of the novel object recognition (ISO-NOR) and fear conditoning tasks, and associated markers such as the downregulation of synaptic plasticity genes and upregulation of neuroinflammation-associated markers.

### (B) Bacterial Strain Growth Conditions

Subsequent bacterial strains cultivation of the Faecalicatena contorta, Lachnospiraceae spp and Ruminococcus lactaris isolates were made in an anerobic chamber and using reduced sterile liquid medium. Initial bacterial seeding of the culture was made from the cryoprotected 16% glycerol bacteria stock described in example 1B. TGV medium was used for bacterial culture and consisted of Tryptone-peptone 3%, Yeast extract 2% (BD Difco), D (+) Glucose 1%, Resazurin sodium salt 2 mg/l, Menadione 0.5 mg/l, Thiamine hydrochloride 1 mg/l, Nicotinic acid 1 mg/l, Riboflavin 0.5 mg/l, p-aminobenzoic acid 0.1 mg/l, Calcium Pantothenate 1 mg/l, Vitamin B12 0.5 ml/l from a 10 ug/ml solution in ultra-pure water, a biotin solution 2.5 ml/l from a 0.2 mg/100 ml solution in ultra-pure water (Sigma), Pyridoxamine dihydrochloride 0.5 mg/l, L-Cysteine hydrochloride monohydrate 0.05%, Folic acid 0.05 mg/l, (Merck) supplemented with a Hemin solution at 2,5% made of Hemin chloride 2 mg/l, Potassium dihydrogen phosphate at 21.76 mg/l (Merck), disodium hydrogen phosphate at 67.2 mg/l (VWR), in ultra-pure water. When bacterial cultures reached the stationary phase, bacteria were centrifugated at 6000g for 5 minutes, resuspended in sterile PBS and centrifugated once more to be aliquoted in 16% glycerol in sterile PBS. The vehicle solution consisted of 16% glycerol in PBS. Samples were stored at −80° C. until use.

(C) Treatment of “Human Alzheimer's Disease-Like Mice” with Purified Bacterial Strains

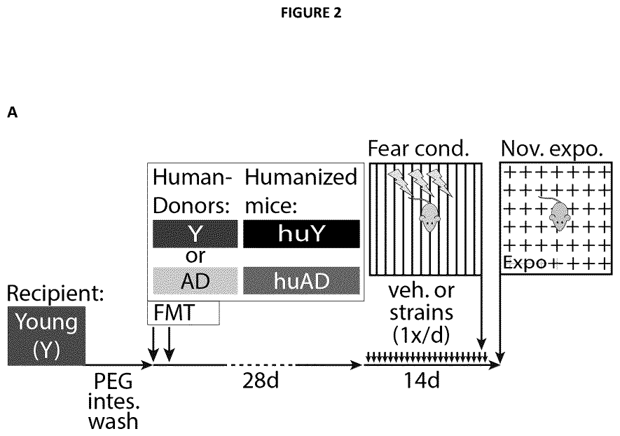

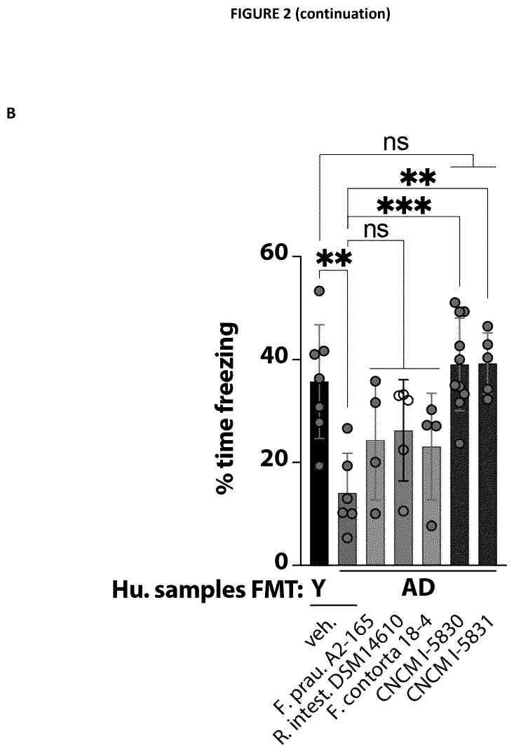

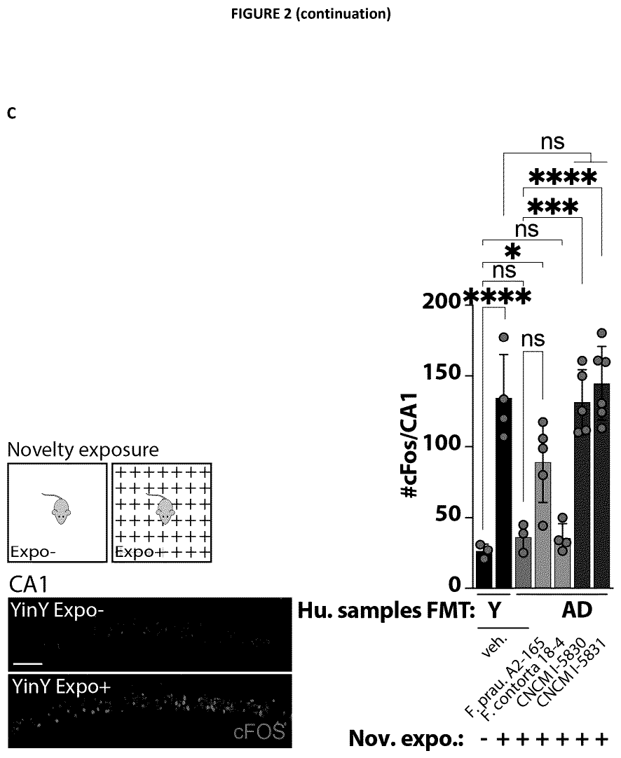

Mice previously established as human Alzheimer's disease-like mice (see above), 28d after the human Alzheimer's disease FMT, received two weeks of either bacterial strain at a dose of 1 to 5×10⁹ viable cells/ml or vehicle treatment by daily gavages at a volume of 300 μl per animal (FIG. 2A). Bacterial strains or vehicle samples (previously stored at −80° C.) were thawed in a 37° C. water bath for 3 min and mice treatment was made through gavage using flexible feeding tubes (FTP-18-38, Instech Laboratories, USA). Control mice consisted in young human FM transferred animals. FMT procedure was identical to the process described above.

### (D) Effect of Treatment on Memory Abilities as Assessed in the Fear Conditioning Task Model

Memory abilities (learning and memory deficits) of the treated mice were determined in the contextual fear conditioning task model, following the protocol from Rei, D. et al., 2015 (Rei et al., 2015). Briefly, on day 12 of the strain treatment, mice were individually placed in the fear apparatus for 3 min before starting a sequence consisting of 3 electrical shocks (0.8 mA for 2s, inter-shock interval of 28s), delivered through the fear conditioning arena's grid floor. Animals were returned to their home cage after 15s. On the next day, the mice were returned to the fear conditioning arena for 3 min and the duration of “freezing”, defined as the absence of any movement except for breathing, was measured by an experimenter blind to the treatment.

Vehicule treated human Alzheimer's disease-like mice (veh., huAD), showed a deterioration of their memory compared to control vehicle young human FM transplanted animals (veh., huY). On the opposite, human Alzheimer's disease-like mice treated with the strains Lachnospiraceae spp CNCM I-5830 (CNCM I-5830, huAD) or Ruminococcus lactaris CNCM 1-5831 (CNCM 1-5831, huAD), showed a total rescue of their memory deficits in the fear conditioning task compared to vehicle huY and vehicle huAD mice (FIG. 2B). Interestingly, other strains such as the Faecalibacterium prausnitzii reference strain A2-165 (F. prau. A2-165, huAD), despite its known anti-inflammatory effect (Bellais et al., 2020), its protective effect in intestinal bowel disorders (IBD) (Martin et al, 2014) and its recent involvment in Alzheimer's disease pathogenesis (Ueda et al., 2021), Roseburia intestinalis strain DSM14610 (R. intest., huAD), despite its known anti-inflammatory effect (Shen et al., 2018), notably through the secretion of butyrate (Duncan et al., 2004), its protective effect in IBD (Luo W. et al, 2019) and its decrease presence in Alzheimer's disease patients (Zhuang et al., 2018), and Faecalicatena contorta 18-4 (F. contorta 18-4, huAD), despite being the highest acetate SCFA producer of our library of strains isolated from young human donors (data not shown), when given as a treatment to human Alzheimer's disease-like mice, showed no improvement in the fear conditioning memory deficits of the treated animals, compared to vehicule human Alzheimer's disease-like (veh., huAD) animals (FIG. 2B). These results show the ability of Lachnospiraceae spp CNCM I-5830 and Ruminococcus lactaris CNCM I-5831 strains to fully restore memory in a model of Alzheimer's disease, as well as the specificity of this effect.

### (E) Effect of Treatment on Memory Abilities as Assessed in the Novelty Exposure Assay

The previous results of the anti-Alzheimer's disease and pro-mnesic effect of Lachnospiraceae spp and Ruminococcus lactaris strains of the invention were confirmed at the hippocampal neuronal network level. Indeed, measure of the ability of the hippocampus to react to the exposure to novelty can be taken as a proxy of the hippocampus mnesia-related neural activity tonus (Rei et al., 2021; Takeuchi et al., 2016). This assessment is made by the measure of the increase in the number of hippocampal neurons positive for the immediate early-gene c-fos, following the exposition of the animal to a novel context. Our previous characterization of the human Alzheimer's disease-like model showed it to be caused by a deficit in the ability of the hippocampus to respond to novelty exposure. Novelty exposure of strain treated human Alzheimer's disease-like model mice was therefore used here as an additional method to assess the anti-Alzheimer's disease/pro mnesic effect of the strains of the invention.

The novelty Exposure assay was done on day 14 of the strain treatment (FIG. 2A) and in accordance to the experimental procedure described in Rei, D., et al., 2021 (Rei et al., 2021). Briefly, animals were exposed to a novel context (an arena with a textured plastic floor) for 3 minutes, before being returned to their home cage. 90 minutes after this novelty exposure, a time corresponding to the pick of c-fos expression following a behavioral stimulus, mice were intracardially perfused with paraformaldehyde, their brains harvested and the number of c-fos positive cells in the hippocampal CA1 subregion was quantified.

In the young human donors FM-transplanted vehicle treated animals, exposure to a novel context (veh., huY, nov.expo.: +), led to an increase in the number of c-fos positive neurons in hippocampal CA1, compared to their non novelty exposed controls (veh., huY, nov.expo.:−), whereas this response to novelty was lost in vehicle treated human Alzheimer's disease-like mice (veh., huAD, nov.expo.: +), compared to vehicle huY animals. Additionnally, treatment of human Alzheimer's disease-like mice with either, the F. Praunitzii A2-165 strain (F. prau A2-165, huAD, nov. expo.: +), or Faecalicatena contorta (F. contorta 18-4, huAD, nov.expo.: +) led to a moderate or to no improvement, respectively, in the number of novelty-exposure induced c-fos activated neurons, when compared to vehicle human Alzheimer's disease-like mice. At the opposite, treatment with the strains Lachnospiraceae spp CNCM I-5830 (CNCM I-5830, huAD, nov.expo.: +), or Ruminococcus lactaris CNCM 1-5831 (CNCM I-5831, huAD, nov.expo.: +), led to a full recovery in the ability of the hippocampus to respond to novelty when compared to vehicle huY (veh., huY, nov.expo.:—/+) and human Alzheimer's disease-like (veh., huAD, nov.expo.: +) animals (FIG. 2C).

In conclusion, it appears that Lachnospiraceae spp and Ruminococcus lactaris according to the invention have the ability to fully restaure memory in a model of Alzheimer's disease.

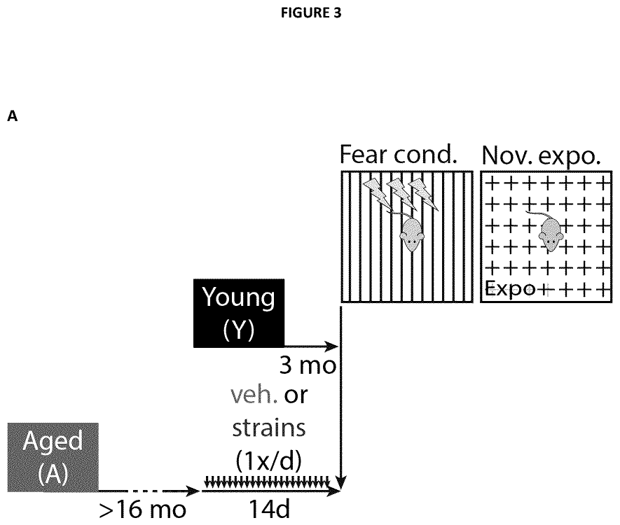

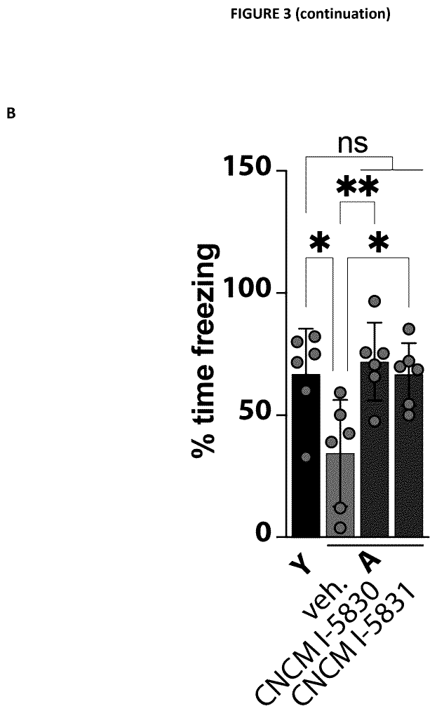

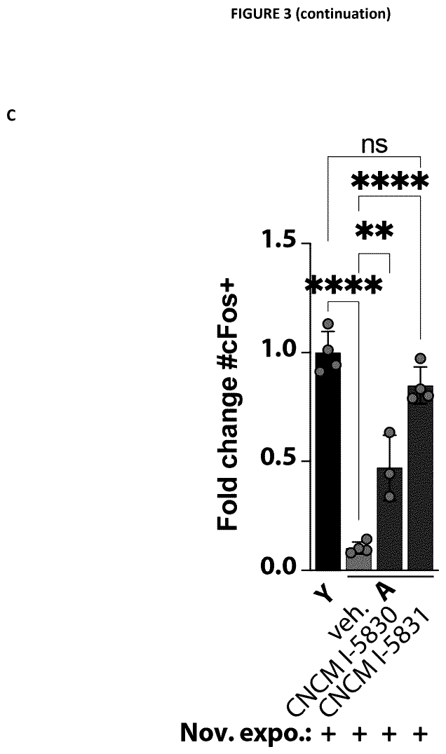

### (F) Effect of Lachnospiraceae Spp CNCM 1-5830 and Ruminococcus Lactaris CNCM I-5831 on Memory Abilities in the Aged Mouse Model of Aging (FIG. 3)

Aged mice were 18 to 20 months male RjOrl: SWISS mice purchased from Janvier labs (St. Berthevin, France). At this age, animals were previously shown to present age-related memory deficits in the ISO-NOL, ISO—NOR and fear conditioning tasks and a complete deficit in the induction of neuronal activation following the exposure to a novel context in the novelty exposure assay (Rei et al., 2021). They also present some changes in memory-deficits related markers such as a decrease in hippocampal neurogenesis and signs of neuroinflammation. This model was therefore used to measure the anti-aging/pro-memory effect of Lachnospiraceae spp CNCM I-5830 and Ruminococcus lactaris CNCM I-5831 of the invention. Control young animals were similar to the mice used as human Alzheimer's disease-like mice controls (10 to 12 weeks old mice).

Bacterial strains isolation and growth conditions, strain treatment, as well as fear conditioning task and novelty exposure assay, were identical to the previous experiments in human Alzheimer's disease-like mice described in example 2 (FIG. 3A).

Vehicle-treated aged (A) mice (veh., A) showed memory deficits in the fear conditioning task model, when compared to vehicle-treated control young (Y) adult mice (veh., Y). The treatment of aged animals with Lachnospiraceae spp CNCM I-5830 (CNCM I-5830, A) and Ruminococcus lactaris CNCM I-5831 (CNCM I-5831, A) led to a complete rescue of their learning memory abilities in comparison to vehicle treated controls (veh., A), as freezing levels of the treated animals in the task were indistinguishable from scores depicted by control young animals (FIG. 3B).

Effect of the strain treatment in aged mice was also evaluated at the hippocampus neuronal network level using the novelty exposure assay. The number of neurons activated by the exposure to a novel context and c-fos positive in the CA1 hippocampal region was drastically diminished in aged mice (veh., A, nov. expo.: +) compared to non-novelty exposed young control animals (Y, nov. expo.:−). Treatment of aged mice with the Lachnospiraceae spp CNCM I-5830 strain (CNCM I-5830, A, nov. expo.: +), led to an increase in the ability of the hippocampus to respond to novelty compared to vehicle treated animals, whereas this ability was totally restored in aged mice treated with the Ruminococcus lactaris CNCM 1-5831 strain (CNCM 1-5831, A, nov. expo.: +), when compared to vehicle treated young and aged control animals (FIG. 3C).

In conclusion, it appears that Lachnoepiraceae spp and Ruminococcus lactaris strains according to the invention revert the memory deficits in a model of Alzheimer's disease and of aging.

### BIBLIOGRAPHIC REFERENCES

Hedden, T., and Gabrieli, J. D. E. (2004). Insights into the ageing mind: a view from cognitive neuroscience. Nat Rev Neurosci 5, 87-96. 10.1038/nrn1323.
Dahan, L., Rampon, C., and Florian, C. (2020). Age-related memory decline, dysfunction of the hippocampus and therapeutic opportunities. Progress in Neuro-Psychopharmacology and Biological Psychiatry 102, 109943. 10.1016/j.pnpbp.2020.109943.
Mufson, E. J., Ikonomovic, M. D., Counts, S. E., Perez, S. E., Malek-Ahmadi, M., Scheff, S. W., and Ginsberg, S. D. (2016). Molecular and cellular pathophysiology of preclinical Alzheimer's disease. Behavioural Brain Research 311, 54-69. 10.1016/j.bbr.2016.05.030.
Holmes A, Finger C, Morales-Scheihing D, Lee J, Mccullough L D. Gut dysbiosis and age-related neurological diseases; an innovative approach for therapeutic interventions. Transl Res. Published online Aug. 2, 2020. doi: 10.1016/j.trsl.2020.07.012
Jeffery I B, Lynch D B, O'Toole P W. Composition and temporal stability of the gut microbiota in older persons. ISME J. 2016; 10 (1): 170-182. doi: 10.1038/ismej.2015.88
Cattaneo A, Cattane N, Galluzzi S, et al. Association of brain amyloidosis with pro-inflammatory gut bacterial taxa and peripheral inflammation markers in cognitively impaired elderly. Neurobiol Aging. 2017; 49:60-68. doi: 10.1016/j.neurobiolaging.2016.08.019
D'Amato A, Di Cesare Mannelli L, Lucarini E, et al. Faecal microbiota transplant from aged donor mice affects spatial learning and memory via modulating hippocampal synaptic plasticity-and neurotransmission-related proteins in young recipients. Microbiome. 2020; 8 (1): 140. doi: 10.1186/s40168-020-00914-w
Li Y, Ning L, Yin Y, et al. Age-related shifts in gut microbiota contribute to cognitive decline in aged rats. Aging. 2020; 12 (9): 7801-7817. doi: 10.18632/aging.103093
Kim N, Jeon S H, Ju I G, et al. Transplantation of gut microbiota derived from Alzheimer's disease mouse model impairs memory function and neurogenesis in C57BL/6 mice. Brain Behav Immun. 2021; 98:357-365. doi: 10.1016/j.bbi.2021.09.002
Rei D, Saha S, Haddad M, et al. Age-Associated Gut Microbiota Impairs Hippocampus-2021:2021.01.28.428594. Dependent Memory in a Vagus-Dependent Manner.; doi: 10.1101/2021.01.28.428594
Sun J, Xu J, Ling Y, et al. Fecal microbiota transplantation alleviated Alzheimer's disease-like pathogenesis in APP/PS1 transgenic mice. Transl Psychiatry. 2019; 9 (1): 1-13. doi: 10.1038/s41398-019-0525-3
Leblhuber F, Steiner K, Schuetz B, Gostner D F and J M. Probiotic Supplementation in Patients with Alzheimer's Dementia—An Explorative Intervention Study. Current Alzheimer Research. Published Oct. 31, 2018. Accessed Nov. 22, 2019.
Macfarlane G T, Macfarlane S. Fermentation in the Human Large Intestine: Its Physiologic Consequences and the Potential Contribution of Prebiotics. J Clin Gastroenterol. 2011; 45: S120. doi: 10.1097/MCG.0b013e31822fecfe
Ohkawara S, Furuya H, Nagashima K, Asanuma N, Hino T. Oral Administration of Butyrivibrio fibrisolvens, a Butyrate-Producing Bacterium, Decreases the Formation of Aberrant Crypt Foci in the Colon and Rectum of Mice. J Nutr. 2005;135 (12): 2878-2883. doi: 10.1093/jn/135.12.2878
Ohkawara S, Furuya H, Nagashima K, Asanuma N, Hino T. Effect of Oral Administration of Butyrivibrio fibrisolvens MDT-1 on Experimental Enterocolitis in Mice. Clin Vaccine Immunol. Published online November 2006. doi: 10.1128/CVI.00267-06
Ohkawara S, Furuya H, Nagashima K, Asanuma N, Hino T. Effect of oral administration of Butyrivibrio fibrisolvens MDT-1, a gastrointestinal bacterium, on 3-methylcholanthrene-induced tumor in mice. Nutr Cancer. 2007; 59 (1): 92-98. doi: 10.1080/01635580701397608
Luan Z, Sun G, Huang Y, et al. Metagenomics Study Reveals Changes in Gut Microbiota in Centenarians: A Cohort Study of Hainan Centenarians. Front Microbiol. 2020; 11:1474. doi: 10.3389/fmicb.2020.01474
Lay C, Sutren M, Rochet V, Saunier K, Dore J, Rigottier-Gois L. Design and validation of 16S rRNA probes to enumerate members of the Clostridium leptum subgroup in human faecal microbiota. Environ Microbiol. 2005;7 (7): 933-946. doi: 10.1111/j.1462-2920.2005.00763.x
Hsiao A, Ahmed A M S, Subramanian S, et al. Members of the human gut microbiota involved in recovery from Vibrio cholerae infection. Nature. 2014; 515 (7527): 423-426. doi: 10.1038/nature13738
Jenq R R, Taur Y, Devlin S M, et al. Intestinal Blautia Is Associated with Reduced Death from Graft-versus-Host Disease. Biol Blood Marrow Transplant J Am Soc Blood Marrow Transplant. 2015;21 (8): 1373-1383. doi: 10.1016/j.bbmt.2015.04.016
Le Roy T, Debedat J, Marquet F, et al. Comparative Evaluation of Microbiota Engraftment Following Fecal Microbiota Transfer in Mice Models: Age, Kinetic and Microbial Status Matter. Front Microbiol. 2019; 9:3289. doi: 10.3389/fmicb.2018.03289
Bellais S, Nehlich M, Duquenoy A, et al. Sorting and cultivation of Faecalibacterium prausnitzii from fecal samples using flow cytometry in anaerobic conditions. bioRxiv. Published online Mar. 25, 2020:2020.03.25.007047. doi: 10.1101/2020.03.25.007047
Rei D, Mason X, Seo J, et al. Basolateral amygdala bidirectionally modulates stress-induced hippocampal learning and memory deficits through a p25/Cdk5-dependent pathway. Proc Natl Acad Sci. 2015;112 (23): 7291-7296. doi: 10.1073/pnas. 1415845112
Ueda A, Shinkai S, Shiroma H, et al. Identification of Faecalibacterium prausnitzii strains for gut microbiome-based intervention in Alzheimer's-type dementia. Cell Rep Med. 2021; 2 (9): 100398. doi: 10.1016/j.xcrm.2021.100398
Shen Z, Zhu C, Quan Y, et al. Insights into Roseburia intestinalis which alleviates experimental colitis pathology by inducing anti-inflammatory responses. J Gastroenterol Hepatol. 2018; 33 (10): 1751-1760. doi: 10.1111/jgh.14144
Duncan S H, Holtrop G, Lobley G E, Calder A G, Stewart C S, Flint H J. Contribution of acetate to butyrate formation by human faecal bacteria. Br J Nutr. 2004; 91 (6): 915-923. doi: 10.1079/BJN20041150
Zhuang Z Q, Shen L L, Li W W, et al. Gut Microbiota is Altered in Patients with Alzheimer's Disease. J Alzheimers Dis. 2018; 63 (4): 1337-1346. doi: 10.3233/JAD-180176
Martin R. et al, The Commensal Bacterium Faecalibacterium prausnitzii Is Protective in DNBS-induced Chronic Moderate and Severe Colitis Models, Inflammatory Bowel Diseases, Volume 20, Issue 3, 1 Mar. 2014, Pages 417-430
Meier-Kolthoff et al. Genome sequence-based species delimitation with confidence intervals and improved distance functions BMC Bioinformatics 2013, 14:60. doi: 10.1186/1471-2105-14-60
Meier-Kolthoff et al. TYGS and LPSN: a database tandem for fast and reliable genome-based classification and nomenclature of prokaryotes. Nucleic Acids Research, Volume 50, Issue D1, 7 Jan. 2022, Pages D801-D807
Meier-Kolthoff et al. Complete genome sequence of DSM 30083T, the type strain (U5/41T) of Escherichia coli, and a proposal for delineating subspecies in microbial taxonomy. Standards in Genomic Sciences volume 9, Article number: 2 (2014)
Luo W. et al, Roseburia intestinalis supernatant ameliorates colitis induced in mice by regulating the immune response. Mol Med Rep. 2019 August; 20 (2): 1007-1016.

1-18. (canceled)

19. A composition comprising Ruminococcus lactaris bacteria strains, and/or a culture extract thereof, and a carrier or excipient, for use in the treatment and/or prevention of memory decline due to ageing or a memory deficit caused by a neuropsychiatric and/or neurodegenerative disease, disorder, or condition.

20. The composition for use of claim 19, wherein the Ruminococcus lactaris bacteria strain is phylogenetically related to Ruminococcus lactaris ATCC 29176.

21. The composition for use of claim 19, wherein the neuropsychiatric and/or neurodegenerative disease, disorder, or condition causing memory deficit is selected from neurodegenerative diseases of the central nervous system, in particular diseases selected from Alzheimer's disease, Parkinson disease, Huntington disease or Amyotrophic Lateral Sclerosis, agnosia, amnesia, traumatic brain injury, dementia, postoperative cognitive dysfunction, or Attention Deficit/Hyperactivity Disorder.

22. The composition for use of claim 19, wherein the neuropsychiatric and/or neurodegenerative disease, disorder, or condition causing memory deficit is Alzheimer's disease.

23. The composition for use of claim 19, comprising the Ruminococcus lactaris bacteria strain having been deposited at the Collection Nationale de Cultures de Microorganismes (CNCM), Institut Pasteur, 25 rue du Docteur Roux, 75724 PARIS Cedex 15, on Mar. 2, 2022, under the accession number CNCM I-5831.

24. The composition for use of claim 19, wherein the culture extract is selected from the group consisting of the strain culture supernatants, cell debris, cell walls, and protein extracts.

25. The composition for use of claim 19, wherein the bacteria or culture extract thereof are in suspension, freeze-dried, spray-dried, either in activated or in inactivated form.

26. The composition for use of claim 19, wherein the bacteria or culture extract are provided as a tablet, capsule, powder, granules or liquid preparations.

27. The composition for use of claim 19, wherein the bacteria or culture extract are formulated for oral administration, preferably as an oral dosage form covered by a coating resistant to gastric juice.

28. The composition for use of claim 19, wherein the bacteria or culture extract are formulated for rectal administration.

29. The composition for use of claim 19, wherein the bacteria or culture extract are formulated as a drug or a LBP drug.

30. The composition for use of claim 19, wherein the bacteria or culture extract are formulated as a probiotic or nutraceutical product.

31. The composition for use of claim 19, wherein the Ruminococcus lactaris bacteria strain is combined with a Lachnospiraceae spp bacteria strain.

32. The composition for use of claim 31, wherein said Lachnospiraceae spp bacteria strain is the Lachnospiraceae spp bacterial strain having been deposited at the Collection Nationale de Cultures de Microorganismes (CNCM), Institut Pasteur, 25 rue du Docteur Roux, 75724 PARIS Cedex 15, on Mar. 2, 2022, under the accession number CNCM I-5830.

33. The composition for use of claim 31, comprising bacteria of the Lachnospiraceae spp bacterial strain having been deposited at the Collection Nationale de Cultures de Microorganismes (CNCM), Institut Pasteur, 25 rue du Docteur Roux, 75724 PARIS Cedex 15, on Mar. 2, 2022, under the accession number CNCM I-5830 and bacteria of the Ruminococcus lactaris strain having been deposited at the Collection Nationale de Cultures de Microorganismes (CNCM), Institut Pasteur, 25 rue du Docteur Roux, 75724 PARIS Cedex 15, on Mar. 2, 2022, under the accession number CNCM I-5831.

34. The composition for use of claim 19, wherein the memory deficit is assessed with one or more object recognition tasks; digit span tasks; motor or spatial memory tasks, such as spatial information tests; mirror drawing tests; mirror reading tasks; weight sampling tasks; speed reading of repeated nonwords; resolving random-dot stereograms; classical conditioning tasks, such as eyeblink conditioning; ability to classify letter strings as grammatical or nongrammatical; fact recall; matching tests of various sorts; tests for retention from minutes to days or years; verbal learning and recall tasks, and combinations of the same.

35. A method of generating a mouse model exhibiting Alzheimer's disease symptoms, said method comprising the step of transferring gut microbiota obtained from fecal samples of Alzheimer-affected human patients to the mouse.

36. A mouse model produced by the method of claim 35.
